(19) United States
(12) Patent Application Publication
(10) Pub. No.: US 2020/0129450 A1
(43) Pub. Date: Apr. 30, 2020

(54) COMPOSITION COMPRISING OSMUNDACETONE OR PHARMACEUTICALLY ACCEPTABLE SALT THEREOF FOR PREVENTING OR TREATING BONE DISEASE

(71) Applicants: KORPHARM CO., LTD., Gyeonggi-do (KR); KOREA UNIVERSITY RESEARCH AND BUSINESS FOUNDATION, Seoul (KR); KOREA INSTITUTE OF RADIOLOGICAL & MEDICAL SCIENCES, Seoul (KR)

(72) Inventors: Gil Hong Park, Seoul (KR); Geu Rim Son, Seoul (KR); Seong Su Hong, Gyeonggi-do (KR); Chun Whan Choi, Gyeonggi-do (KR); Yun Hyeok Choi, Gyeonggi-do (KR); Woo Jung Kim, Gyeonggi-do (KR); Han Kyeom Kim, Seoul (KR); Jae Woo Kang, Seoul (KR); Eun Jung Lee, Gyeonggi-do (KR); Serk In Park, Seoul (KR); Kee Ho Lee, Seoul (KR); Min Ji Choi, Gyeonggi-do (KR)

(21) Appl. No.: 16/338,354

(22) PCT Filed: Sep. 28, 2017
(86) PCT No.: PCT/KR2017/010822

§ 371 Date: Jun. 14, 2019

(30) Foreign Application Priority Data
Sep. 30, 2016 (KR) 10-2016-0126767
Oct. 28, 2016 (KR) 10-2016-0142422

(51) Int. Cl. A61K 31/12; A61K 36/12; A61P 19/08

(52) U.S. Cl. CPC A61K 31/12; A61K 2236/333; A61P 19/08; A61K 36/12

(57) ABSTRACT

The present invention relates to a composition comprising osmundacetone or a pharmaceutically acceptable salt thereof for preventing or treating bone diseases. More specifically, the present invention relates to: a composition comprising osmundacetone, a pharmaceutically acceptable salt thereof, or an Osmunda japonica extract as an effective ingredient, for preventing or treating osteoporosis, rheumatoid arthritis, arthralgia, Paget disease, bone metastatic cancer, or fracture; a food composition for improvement; a use of a salt; and a treatment method. The composition according to the present invention shows a strong inhibitory activity against proliferation and differentiation of osteoclast which causes bone loss, and activation of differentiation of osteoblast, and thus can be usefully utilized in developing medicines for safe and effective treatment of bone diseases or functional foods for improving symptoms of bone diseases

The present application is a national stage of PCT/KR2017/010822, filed Sep. 28, 2017, which claims priority from Korean Patent Application No. 10-2016-0126767 filed on Sep. 30, 2016 and Korean Patent Application No. 10-2016-0142422 filed on Oct. 28, 2016, the disclosures of which are incorporated herein by reference in their entities.

### TECHNICAL FIELD

The present invention relates to a composition comprising osmundacetone or a pharmaceutically acceptable salt thereof for the prevention or treatment of a bone disease and, more specifically, to a pharmaceutical composition for the prevention or treatment of a bone disease and a food composition for alleviating a bone disease, each of the composition containing osmundacetone or a pharmaceutically acceptable salt thereof or an Osmunda japonica extract as an active ingredient, to a use for preparing an agent for the prevention or treatment of a bone disease, and a method for the treatment of a bone disease.

### BACKGROUND ART

Bone plays a very important role in forming the skeletal structure of the body and maintaining blood calcium (Ca²⁺) levels. Bone is maintained through a balanced bone remodeling cycle between osteoclasts that metabolically resorb bones and osteoblasts that form bones. When the amount of bone uptake is greater than the amount of bone formation due to the destruction of the balance between bone resorption and formation, a variety of bone-related diseases occur. Representative diseases associated with differentiation and activation of osteoclasts may include osteoporosis, rheumatoid arthritis, joint pain, Paget's disease, bone metastatic cancer, and bone fractures (Kim J H and Kim N, 2016; Shiozawa Y et al., 2011; and Singer F R, 2016).

Out of these, osteoporosis is caused when the amount of bone uptake is greater than the amount of bone formation since the balance between bone resorption and formation is destroyed due to the activation of osteoclasts. In osteoporosis, the density of bone parenchyma decreases and thus the frequency of bone fractures increases.

Osteoporosis most frequently occurs in women, such as middle-aged and elderly women, who have hormonal imbalance, and also occurs in patients who cannot move due to fractures or severe disease. Recently, the incidence of osteoporosis is increasing in even middle-aged or elderly men.

The following two cytokines play an important role in the molecular mechanism by which bone marrow macrophage/monocyte lineage cells differentiate into osteoclasts (Teitelbaum S L and Ross F P, 2003). (i) When macrophage colony-stimulating factor (M-CSF) binds to its receptor c-Fms, osteoclast progenitor cells proliferate and survive. When receptor activator of nuclear factor-KB ligand (RANKL) binds to its receptor RANK, osteoclast differentiation and bone resorption are activated and mature osteoclasts survive (Lacey D L et al., 1998; Lum L et al., 1999; Sherr C J, 1990; Suda T et al., 1999; and Wong B R et al., 1999). (ii) When M-CSF induces the activation of c-Fms, osteoclast progenitor cells proliferate and survive via ERK and PI3K/Akt pathways (Mancini et al., 1997). (iii) RANKL (OPGL, ODF, TRANCE) and RANK also control the formation and functions of osteoclasts (Anderson D M et al., 1997; Dougall W C et al., 1999; and Kong Y Y et al., 1999). When RANKL binds to RANK, TNF receptor-associated factors (TRAFs), such as TRAFs 1, 2, 3, 5, and 6, bind to RANK (Darnay B G et al., 1998; and Walsh M C and Choi Y, 2003). Out of these, TRAF6 is most important in the formation and functions of osteoclasts (Lomaga M A et al., 1999; and Naito A et al., 1999). TRAF6 delivers RANKL/RANK signals NF-κB, c-Jun N-terminal kinase (JNK), extracellular signal-regulated kinase (ERK), p38, Akt, Nuclear Factor Of Activated T-Cells 1 (NFATc1) to induce osteoclast proliferation, fusion, and differentiation (Kobayashi N et al., 2001; Lomaga M A et al., 1999; Naito A et al., 1999; Takayanagi H et al., 2002; Wong B R et al., 1998; and Wong B R et al., 1999).

Existing directions of development of osteoporosis medicines were to identify substances capable of preventing the loss of bone parenchyma by suppressing bone resorption of osteoclasts. The representative drug is bisphosphonate family Fosamax. In the same context, much research has been conducted on effects of arachidonate metabolites on bone tissue metabolism (Lee Sung-eun, 1999). Leukotriene-B4 (LTB4) is one of metabolites of 5-lipoxygenase pathway, which is a metabolic pathway of arachidonate (Ford-Hutchinson, A. W. et al., 1980). C433, which are interstitial cells obtained from the giant cell tumor, has been reported to increase the number and activity of osteoblasts by increasing 5-lipoxygenase metabolites (Mundy, G. R. et al., 1993). It was observed that the administration of LTB4 during bone tissue culture increased bone resorption (Bonewald, L. F. et al., 1996). In vitro and in vivo studies also showed that LTB4 increases the production of osteoclasts to induce bone resorption (Bonewald, L. F. et al., 1996). Hence, LTB4 receptor antagonists have been developed for the treatment of osteoporosis, but such antagonists did not succeed in sufficiently suppressing the bone parenchyma resorption of osteoclasts.

Moreover, side effects of existing osteoporosis medicines and high prices thereof are also a major obstacle to administering such osteoporosis medicines in sufficient doses for the treatment of patients. Main side effects of Fosamax include severe esophagitis, renal damage, liver damage, hypocalcemia, muscle spasms, and the like, and Roche's Bonviva has side effects, such as systemic muscle aches and body aches. Aclasta (zoledronate) by Novartis and Forsteo and Forteo (teriparatide), which are parathyroid hormones, as anabolic medicines, by Eli Lilly, are very effective, but are very restricted in use due to too high prices thereof. Especially, Forsteo/Forteo cannot be used for patients who are pregnant or breastfeeding, patents who have drug hypersensitivity, metabolic bone diseases, such as hypercalcemia, kidney failure, hyperparathyroidism, and Paget's disease, unexplained elevations of alkaline phosphatase, patients undergoing radiotherapy, or patients having bone marrow cancer or bone metastatic cancer, and thus the applicable patient group thereof is not large.

Therefore, the development of bone disease-related medicines that are more effective and safer and can be produced at lower cost compared with existing medicines is urgently needed.

### DETAILED DESCRIPTION OF THE INVENTION

### Technical Problem

The present inventors studied natural substance components in order to develop bone-related disease medicines having few side effects, being safe, and showing excellent effects, and as a result, the present inventors verified that an extract of Osmunda japonica that has been for a food for a long time has bone loss inhibitory activity, and thus completed the present invention.

Therefore, an aspect of the present invention is to provide a pharmaceutical composition for preventing or treating a bone disease, the composition comprising osmundacetone or a pharmaceutically acceptable salt thereof as an active ingredient, wherein the bone disease is at least one selected from the group consisting of osteoporosis, rheumatoid arthritis, arthralgia, Paget's disease, bone metastatic cancer, and bone fractures.

Another aspect of the present invention is to provide a food composition for preventing or alleviating a bone disease, the composition comprising osmundacetone or a pharmaceutically acceptable salt thereof as an active ingredient, wherein the bone disease is at least one selected from the group consisting of osteoporosis, rheumatoid arthritis, arthralgia, Paget's disease, bone metastatic cancer, and bone fractures.

Still another aspect of the present invention is to provide a pharmaceutical composition for preventing or treating a bone disease, the composition comprising an Osmunda japonica extract as an active ingredient, wherein the bone disease is at least one selected from the group consisting of osteoporosis, rheumatoid arthritis, arthralgia, Paget's disease, bone metastatic cancer, and bone fractures.

Still another aspect of the present invention is to provide a food composition for alleviating a bone disease, the composition comprising an Osmunda japonica extract as an active ingredient, wherein the bone disease is at least one selected from the group consisting of osteoporosis, rheumatoid arthritis, arthralgia, Paget's disease, bone metastatic cancer, and bone fractures.

Still another aspect of the present invention is to provide a use of osmundacetone or a pharmaceutically acceptable salt thereof for the preparation of an agent for preventing or treating a bone disease, wherein the bone disease is at least one selected from the group consisting of osteoporosis, rheumatoid arthritis, arthralgia, Paget's disease, bone metastatic cancer, and bone fractures.

Still another aspect of the present invention is to provide a method for treating a bone disease in a subject, the method comprising administering an effective amount of osmundacetone or a pharmaceutically acceptable salt thereof to a subject in need thereof, wherein the bone disease is at least one selected from the group consisting of osteoporosis, rheumatoid arthritis, arthralgia, Paget's disease, bone metastatic cancer, and bone fractures.

Still another aspect of the present invention is to provide a use of an Osmunda japonica extract for the preparation of an agent for preventing or treating a bone disease, wherein the bone disease is at least one selected from the group consisting of osteoporosis, rheumatoid arthritis, arthralgia, Paget's disease, bone metastatic cancer, and bone fractures.

Still another aspect of the present invention is to provide a method for treating a bone disease in a subject, the method comprising administering an effective amount of an Osmunda japonica extract to a subject in need thereof, wherein the bone disease is at least one selected from the group consisting of osteoporosis, rheumatoid arthritis, arthralgia, Paget's disease, bone metastatic cancer, and bone fractures.

### Technical Solution

In accordance with an aspect of the present invention, there is provided a pharmaceutical composition for preventing or treating a bone disease, the composition comprising osmundacetone or a pharmaceutically acceptable salt thereof as an active ingredient, wherein the bone disease is at least one selected from the group consisting of osteoporosis, rheumatoid arthritis, arthralgia, Paget's disease, bone metastatic cancer, and bone fractures.

In accordance with an aspect of the present invention, there is provided a pharmaceutical composition for preventing or treating a bone disease, the composition consisting of osmundacetone or a pharmaceutically acceptable salt thereof as an active ingredient, wherein the bone disease is at least one selected from the group consisting of osteoporosis, rheumatoid arthritis, arthralgia, Paget's disease, bone metastatic cancer, and bone fractures.

In accordance with an aspect of the present invention, there is provided a pharmaceutical composition for preventing or treating a bone disease, the composition essentially consisting of osmundacetone or a pharmaceutically acceptable salt thereof as an active ingredient, wherein the bone disease is at least one selected from the group consisting of osteoporosis, rheumatoid arthritis, arthralgia, Paget's disease, bone metastatic cancer, and bone fractures.

In accordance with another aspect of the present invention, there is provided a food composition for preventing or alleviating a bone disease, the composition comprising osmundacetone or a pharmaceutically acceptable salt thereof as an active ingredient, wherein the bone disease is at least one selected from the group consisting of osteoporosis, rheumatoid arthritis, arthralgia, Paget's disease, bone metastatic cancer, and bone fractures.

In accordance with another aspect of the present invention, there is provided a food composition for preventing or alleviating a bone disease, the composition consisting of osmundacetone or a pharmaceutically acceptable salt thereof as an active ingredient, wherein the bone disease is at least one selected from the group consisting of osteoporosis, rheumatoid arthritis, arthralgia, Paget's disease, bone metastatic cancer, and bone fractures.

In accordance with another aspect of the present invention, there is provided a food composition for preventing or alleviating a bone disease, the composition consisting essentially of osmundacetone or a pharmaceutically acceptable salt thereof as an active ingredient, wherein the bone disease is at least one selected from the group consisting of osteoporosis, rheumatoid arthritis, arthralgia, Paget's disease, bone metastatic cancer, and bone fractures.

In accordance with still another aspect of the present invention, there is provided a pharmaceutical composition for preventing or treating a bone disease, the composition comprising an Osmunda japonica extract as an active ingredient, wherein the bone disease is at least one selected from the group consisting of osteoporosis, rheumatoid arthritis, arthralgia, Paget's disease, bone metastatic cancer, and bone fractures.

In accordance with still another aspect of the present invention, there is provided a pharmaceutical composition for preventing or treating a bone disease, the composition consisting of an Osmunda japonica extract as an active ingredient, wherein the bone disease is at least one selected from the group consisting of osteoporosis, rheumatoid arthritis, arthralgia, Paget's disease, bone metastatic cancer, and bone fractures.

In accordance with still another aspect of the present invention, there is provided a pharmaceutical composition for preventing or treating a bone disease, the composition consisting essentially of an Osmunda japonica extract as an active ingredient, wherein the bone disease is at least one selected from the group consisting of osteoporosis, rheumatoid arthritis, arthralgia, Paget's disease, bone metastatic cancer, and bone fractures.

In accordance with still another aspect of the present invention, there is provided a food composition for preventing or alleviating a bone disease in a subject, the composition comprising an Osmunda japonica extract as an active ingredient, wherein the bone disease is at least one selected from the group consisting of osteoporosis, rheumatoid arthritis, arthralgia, Paget's disease, bone metastatic cancer, and bone fractures.

In accordance with still another aspect of the present invention, there is provided a food composition for preventing or alleviating a bone disease in a subject, the composition consisting of an Osmunda japonica extract as an active ingredient, wherein the bone disease is at least one selected from the group consisting of osteoporosis, rheumatoid arthritis, arthralgia, Paget's disease, bone metastatic cancer, and bone fractures.

In accordance with still another aspect of the present invention, there is provided a food composition for preventing or alleviating a bone disease in a subject, the composition consisting essentially of an Osmunda japonica extract as an active ingredient, wherein the bone disease is at least one selected from the group consisting of osteoporosis, rheumatoid arthritis, arthralgia, Paget's disease, bone metastatic cancer, and bone fractures.

In accordance with still another aspect of the present invention, there is provided a use of osmundacetone or a pharmaceutically acceptable salt thereof for the preparation of an agent for preventing or treating a bone disease, wherein the bone disease is at least one selected from the group consisting of osteoporosis, rheumatoid arthritis, arthralgia, Paget's disease, bone metastatic cancer, and bone fractures.

In accordance with still another aspect of the present invention, there is provided a method for treating a bone disease in a subject, the method comprising administering an effective amount of osmundacetone or a pharmaceutically acceptable salt thereof to a subject in need thereof, wherein the bone disease is at least one selected from the group consisting of osteoporosis, rheumatoid arthritis, arthralgia, Paget's disease, bone metastatic cancer, and bone fractures.

In accordance with still another aspect of the present invention, there is provided a use of an Osmunda japonica extract for the preparation of a preparation for preventing or treating a bone disease in a subject, wherein the bone disease is at least one selected from the group consisting of osteoporosis, rheumatoid arthritis, arthralgia, Paget's disease, bone metastatic cancer, and bone fractures.

In accordance with still another aspect of the present invention, there is provided a method for treating a bone disease in a subject, the method comprising administering an effective amount of an Osmunda japonica extract to a subject in need thereof, wherein the bone disease is at least one selected from the group consisting of osteoporosis, rheumatoid arthritis, arthralgia, Paget's disease, bone metastatic cancer, and bone fractures.

Hereinafter, the present invention will be described in detail.

The present invention provides a pharmaceutical composition for preventing or treating a bone disease, the composition comprising osmundacetone or a pharmaceutically acceptable salt thereof as an active ingredient, wherein the bone disease is at least one selected from the group consisting of osteoporosis, rheumatoid arthritis, arthralgia, Paget's disease, bone metastatic cancer, and bone fractures.

The pharmaceutical composition according to the present invention may be a composition comprising osmundacetone as an active ingredient, a composition consisting of osmundacetone as an active ingredient, or a composition consisting essentially of osmundacetone as an active ingredient.

As used herein, the term “comprising” is used synonymously with “containing (including)” or “characterized by”, and does not exclude specifically unrecited and additional ingredients or method steps in the compositions and methods according to the present invention. The term “consisting of” is meant to exclude additional elements, steps, or ingredients that are not otherwise indicated. The term “consisting essentially of” is meant to include not only described materials or steps but also any material or step that does not substantially affect basic characteristics thereof in the scope of a composition or method.

<Structure of Osmundacetone>

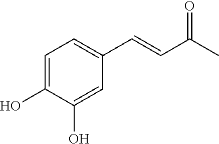

Osmundacetone is a compound, which is represented by the molecular weight C₁₀H₁₀O₃ (molecular weight: 178.184 Da) and has a structure of the above chemical formula. Also, osmundacetone is an ingredient that is isolated and identified from an Osmunda japonica extract by the present inventors and has ability to inhibit the proliferation and differentiation of osteoclasts and activate the differentiation of osteoblasts. Alternatively, osmundacetone is called dihydroxybenzylideneacetone, (3E)-4-(3,4-dihydroxyphenyl)-3-buten-2-one, or by the IUPAC name, 5,7-dihydroxy-2-(4-hydroxyphenyl)-8-[(2S, 3R, 4R, 5S, 6R)-3,4,5-trihydroxy-6-(hydroxymethyl)oxan-2-yl]-6-[(2S, 3R, 4S, 5S)-3,4,5-trihydroxyoxan-2-yl]-4H-chromen-4-one, and present as colorless or yellow to brown colored crystals at room temperature.

Osmundacetone was determined as being harmless and safe in all the tests of human toxicity, irritation, carcinogenicity, environmental toxicity, and the like by a number of US toxic substance regulatory agencies (Note: TSCA: Not Listed; CLEAN WATER ACT (CWA): Not Listed; SARA313: Not Listed; MARINE POLLUTANT: Not Listed; RIGHT TO KNOW LIST (NEW JERSEY): Not Listed; RIGHT TO KNOW LIST (MASSACHUSETTS): Not Listed; RIGHT TO KNOW LIST (PENNSYLVANIA): Not Listed; ILLINOIS TOXIC AIR CONTAMINANTS: Not Listed; CLEAN AIR ACT (CAA): Not Listed; DHS CHEMICALS OF INTEREST: Not Listed; CALIFORNIA PROP 65: Not Listed; OSHA: Not Listed; CALIFORNIA PROP 65 TOXICITY TYPE (CANCER, DEVELOPMENTAL, FEMALE, MALE): None; OSHA HAZ CLASS(CARCINOGEN, CORROSIVE, FLAMMABLE, REACTIVE, TOXIC): None, and the like).

The osmundacetone contained in the composition of the present invention may be used as osmundacetone per se or in the form of a salt, preferably a pharmaceutically acceptable salt. As used herein, the term “pharmaceutically acceptable” refers to being physiologically acceptable, and not usually causing an allergic response or a similar response when administered to a human being. An acid addition salt formed by a pharmaceutically acceptable free acid is preferable as the salt. An inorganic acid and an organic acid may be used as the free acid. Examples of the organic acid include, but are not limited to, citric acid, acetic acid, lactic acid, tartaric acid, maleic acid, fumaric acid, formic acid, propionic acid, oxalic acid, tripleuroacetic acid, benzoic acid, gluconic acid, meta sulfonic acid, glycolic acid, succinic acid, 4-toluenesulfonic acid, glutamic acid, and aspartic acid. Examples of the inorganic acid include, but are not limited to, hydrochloric acid, bromic acid, sulfuric acid, and phosphoric acid.

The osmundacetone may be chemically synthesized, or isolated from a natural substance. When osmundacetone isolated from a natural substance is used, such osmundacetone may be isolated from, especially, a plant in the family Osmundaceae. Most preferably, the osmundacetone may be isolated from Osmunda japonica belongs to the family Osmundaceae.

The plants of the family Osmundaceae are plants of the only one family that belongs to the order Osmundales. The order Osmundales are a group of old ferns originating from the Triassic period of the Mesozoic Era, about 210 million years ago, and classified into leptosporangiate ferns.

Osmunda japonica belonging to the family Osmundaceae has a scientific name of Osmunda japonica Thunb. or Osmunda nipponica Makino, and a Latin name of Osmundae Rhizoma. Osmunda japonica is a plant also called Japanese royal fern or Japanese flowering fern in English, and grows wild in East Asia, such as Japan, China, Korea, and Taiwan, and Russia. Osmunda japonica is a medicinal plant since young leaves thereof have been used not only as food materials for a long time and but also for various diseases through traditional medicines and folk remedies. Osmunda japonica contains osmundacetone, osmundacetone, osmundalin, dihydroisoomundalin, parasorboside, and molting hormones, such as ponasterone A, ecdysone, and ecdysterone. In traditional medicines, roots and stems of Osmunda japonica are called jagi or Jagigwanjung, and are poisonous, but used for the extermination of roundworms, tapeworms, threadworms, and the like; insecticidal effects including antiviral or antibacterial effects; heat dissipating and detoxifying; stopping blood by removing blood stagnation; and treatments for cold caused by wind and heat, skin rashes caused by epidemic febrile, blood vomiting, nasal bleeding, hemafecia caused by internal hemorrhoids, dysentery, leucorrhea, and the like (The encyclopedia of oriental herbal medicine).

The present inventors verified from an Example that an Osmunda japonica extract effectively inhibited the differentiation of osteoclasts that function to destruct and resorb bone tissues. Mononuclear cells as stem cell precursor cells of osteoclasts were isolated from bone marrow cells isolated from mice, stimulated with RANKL and M-CSF as differentiation promoting factors, and treated with an Osmunda japonica extract, and thus the effect of the extract on osteoclast differentiation was examined. As a result, it was verified that a hot-water extract or ethyl acetate extract of the Osmunda japonica extract effectively inhibited the differentiation of bone marrow cells into multinucleated osteoclasts.

The present inventors isolated and identified an ingredient, which has effects of inhibiting osteoclast differentiation and activating osteoblast differentiation, from an Osmunda japonica extract by using HPLC and NMR. Osmundacetone is a single compound that is isolated and identified from a hot-water extract and an ethyl acetate extract/fraction of Osmunda japonica. Osmundacetone has not only excellent osteoclast differentiation inhibitory activity and osteoblast activating activity, but is also safe due to very low cytotoxicity.

Therefore, a person skilled in the art can understand that effective prevention, alleviation of symptoms, or treatment of various bone diseases caused by reductions in bone density and strength resulting from the destruction of the balance between bone resorption by osteoclasts and formation of new bone matrix by osteoblasts and the balance of the bone metabolic process during subsequent mineralization can be expected by using the above activities of the Osmunda japonica extract and osmundacetone, established by the present inventors.

Preferably, the bone disease herein may be osteoporosis, rheumatoid arthritis, arthralgia, Paget's disease, bone metastatic cancer, or a bone fracture, and the correlations between the respective diseases and osteoclasts will be described with reference to application examples of the present specification.

As used herein, the term “treatment” or “treating” means a clinical procedure intended to alter a natural course of an individual or cell to be treated, and the treatment may also be performed for the prevention of clinical pathology. Preferable effects of the treatment include suppressing occurrence or recurrence of disease, relieving symptoms, reducing direct or indirect pathological consequences of disease, reducing disease progression rates, improving, bettering, or relieving disease conditions, or improving prognosis. As used herein, the term “prevention” or “preventing” refers to all actions that suppress the occurrence of diseases or delays the progress of disease.

As for a dose of the pharmaceutical composition of the present invention, an appropriate effective amount thereof may be determined according to the foregoing particular uses by a person skilled in the art considering various factors, such as the route of administration, the time of administration, the number of times of treatment, the period of treatment, and the age, weight, health condition, sex, severity of disease, susceptibility to drugs, diet, and excretion rate of a subject in need of treatment. The term “effective amount” refers to the amount sufficient to show effects of alleviating, treating, preventing, detecting, or diagnosing a bone disease when administered to a subject. The term “subject” may be an animal, preferably a mammal, and more preferably, an animal including a human being, and may be cells, a tissue, an organ, or the like, derived from an animal. The subject may be a bone disease patient in need of treatment.

The administration may be performed once a day or divided into several times. The pharmaceutical composition of the present invention may be administered alone or co-administered with another therapeutic agent known to have effects on the prevention or treatment of a bone disease. In a case of the co-administration, the pharmaceutical composition and another therapeutic agent may be administered sequentially or simultaneously. The dose of the pharmaceutical composition of the present invention when administered alone or in combination is preferably such that the maximum effect can be obtained in a minimal amount without side effects, and such an amount can be easily determined by a person skilled in the art.

A total effective amount of the pharmaceutical composition of the present invention may be administered to a patient in a single dose, or may be administered in multiple doses for a long period of time by a fractionated treatment protocol. In the pharmaceutical composition of the present invention, the content of the active ingredient may vary depending on the severity of disease.

A total dosage of the pharmaceutical composition of the present invention may be preferably about 0.01 μg to 10,000 mg, and more preferably 0.1 μg to 500 mg relative to 1 kg of patient body weight per day. As for the dosage of the pharmaceutical composition, an effective dose thereof to a patient is determined considering various factors, such as the method for formulation, route of administration, number of times of treatment, and the age, weight, health condition, sex, severity of disease, diet, and excretion rate of the patient, and thus considering these factors, a person skilled in the art could determine a proper effective dose of the composition of the present invention. The pharmaceutical composition according to the present invention is not particularly limited to the dosage form, route of administration, and administration method thereof.

The pharmaceutical composition of the present invention may be variously formulated, together with a pharmaceutically acceptable carrier, according to the route of administration, by a method known in the art. The term “pharmaceutically acceptable” composition refers to a non-toxic composition that is physiologically acceptable, does not inhibit an action of an active ingredient when administered to a human being, and does not usually cause an allergic reaction or similar reactions, such as gastroenteric troubles and dizziness. The carrier includes all kinds of solvents, dispersion media, oil-in-water or water-in-oil emulsions, aqueous compositions, liposomes, microbeads, and microsomes.

The route of administration may be an oral or parenteral route. The parental administration may be, but is not limited to, intravenous, intramuscular, intra-arterial, intramedullary, intradural, intracardiac, transdermal, subcutaneous, intraperitoneal, intranasal, intestinal, topical, sublingual, or rectal administration.

The pharmaceutical composition of the present invention, when orally administered, may be formulated, together with a suitable carrier for oral administration, in the form of a powder, granules, a tablet, a pill, a sugar coated tablet, a capsule, a liquid, a gel, a syrup, a suspension, a wafer, or the like, by a method known in the art. Examples of the suitable carrier may include: saccharides including lactose, dextrose, sucrose, sorbitol, mannitol, xylitol, erythritol, and maltitol; starches including corn starch, wheat starch, rice starch, and potato starch; celluloses including cellulose, methyl cellulose, sodium carboxy methyl cellulose, and hydroxypropyl methyl cellulose; and fillers, such as gelatin and polyvinyl pyrrolidone. In some cases, cross-linked polyvinyl pyrrolidone, agar, alginic acid, sodium alginate, or the like may be added as a disintegrant. Furthermore, the pharmaceutical composition may further contain an anti-coagulant, a lubricant, a wetting agent, an aroma, an emulsifier, a preservative, and the like.

As for the parenteral administration, the pharmaceutical composition of the present invention may be formulated in the form of an injection, a transdermal administration preparation, and a nasal inhalant, together with a suitable parenteral carrier, by a method known in the art. The injection needs to be essentially sterilized, and needs to be protected from the contamination of microorganisms, such as bacteria and fungus. Examples of the suitable carrier for the injection may be a solvent or a dispersion medium, including water, ethanol, a polyol (e.g., glycerol, propylene glycol, liquid polyethylene glycol, etc.), a mixture thereof, and/or vegetable oil, but are not limited thereto. More preferably, the suitable carrier may be an isotonic solution, such as Hank's solution, Ringer's solution, phosphate buffered saline (PBS) containing triethanol amine or sterilized water for injection, 10% ethanol, 40% propylene glycol, and 5% dextrose. In order to protect the injection against microbial contamination, the injection may further contain various antimicrobial and antifungal agents, such as paraben, chlorobutanol, phenol, sorbic acid, and thimerosal. In most cases, the injection may further contain an isotonic agent, such as sugar or sodium chloride.

The dosage form of the transdermal administration preparation includes an ointment, a cream, a lotion, a gel, a solution for external application, a paste, a liniment, and an aerosol. The “transdermal administration” means the delivery of an effective amount of an active ingredient contained in the pharmaceutical composition into the skin by the local administration of the pharmaceutical composition into the skin. For example, the pharmaceutical composition of the present invention may be prepared into an injection formulation, which is then administered by slight pricking of the skin using a 30-gauge needle or direct application to the skin. These formulations are described in the literature, which is a formulary generally known in pharmaceutical chemistry (Remington's Pharmaceutical Science, 15th Edition, 1975, Mack Publishing Company, Easton, Pa.).

As for an inhalational administration preparation, the compound used according to the present invention may be conveniently delivered in the form of an aerosol spray from a pressurized pack or a nebulizer by using a suitable propellant, such as dichlorofluoromethane, trichlorofluoromethane, dichlorotetrafluoroethane, or carbon dioxide, or another suitable gas. As for a pressurized aerosol, the dose unit may be determined by providing a valve that delivers a metered amount. For example, a gelatin capsule and a cartridge used in an inhaler or an insufflator may be formulated to contain a powder mixture of a compound and a suitable powder base material, such as lactose or starch.

Other pharmaceutically acceptable carriers may be referenced in the following literature (Remington's Pharmaceutical Sciences, 19th ed., Mack Publishing Company, Easton, Pa., 1995).

The pharmaceutical composition according to the present invention may further contain at least one buffer (for example, saline solution or PBS), a carbohydrate (for example, glucose, mannose, sucrose, or dextran), an antioxidant, a bacteriostat, a chelating agent (for example, EDTA or glutathione), an adjuvant (for example, aluminum hydroxide), a suspending agent, a thickener, and/or a preservative).

The pharmaceutical composition of the present invention may also be formulated by a method known in the art so as to provide rapid, continuous, or delayed release of an active ingredient after administration to a mammal.

Furthermore, the present invention provides a food composition for preventing or alleviating a bone disease, the composition comprising osmundacetone or a pharmaceutically acceptable salt thereof as an active ingredient, wherein the bone disease is at least one selected from the group consisting of osteoporosis, rheumatoid arthritis, arthralgia, Paget's disease, bone metastatic cancer, and bone fractures.

The effects of osmundacetone on the prevention or alleviation of the bone diseases, established by the present inventors, are as described in the present specification.

The food composition includes all types including functional food, nutritional supplements, health food, food additives, and the like. The above types may be prepared into various forms according to the conventional methods known in the art.

For example, for the health food, the food composition itself of the present invention may be drunken by preparation in the form of tea, juice, and drink, or may be taken by granulation, capsulation, or powdering. The food composition of the present invention may be prepared into a form of a composition by mixing with a known substance or active ingredient, which is known to have an effect of prevention or alleviation of a bone disease.

Also, the functional food may be manufactured by adding the food composition of the present invention to beverages (including alcoholic beverages), fruits and processed foods thereof (e.g., canned fruit, bottled food, jam, marmalade, etc.), fishes, meats and processed foods thereof (e.g., ham, sausage, corned beef, etc.), breads, noodles (e.g., udong, buckwheat noodles, ramen, spaghetti, macaroni, etc.), fruit juices, a variety of drinks, cookies, syrups, dairy products (e.g., butter, cheese, etc.), edible vegetable oils, margarine, vegetable proteins, retort foods, frozen foods, and various seasonings (e.g., soybean paste, soybean sauce, sauces, etc.).

A preferable content of the food composition according to the present invention is 0.01-50 wt % relative to a total weight of the finally manufactured food, but is not limited thereto. In order to use the food composition of the present invention in the form of a food additive, the food composition may be prepared in the form of a powder or a concentrate.

Furthermore, the present invention provides a pharmaceutical composition for preventing or treating a bone disease, the composition comprising an Osmunda japonica extract as an active ingredient, wherein the bone disease is at least one selected from the group consisting of osteoporosis, rheumatoid arthritis, arthralgia, Paget's disease, bone metastatic cancer, and bone fractures.

The expected effects of the Osmunda japonica extract on the prevention or alleviation of bone diseases, established by the present inventors, are as described in the present specification. Especially, it has been established that the compound, osmundacetone, contained in the Osmunda japonica extract, has ability to inhibit the proliferation and differentiation of osteoclasts responsible for bone resorption and activate the differentiation of osteoblasts.

The Osmunda japonica extract may be prepared from fresh Osmunda japonica, and Osmunda japonica having passed through processing procedures for storage, such as freezing or drying, may be used. The Osmunda japonica extract is not restricted for morphology or properties thereof, and may be a solution or a concentrate, or a solid or a powder obtained by removing a solvent used in the preparation of the extract.

The Osmunda japonica extract can be used without limitation as long as the extract is known to be obtained by a natural product extraction method. Especially, an extraction method capable of preparing an extract containing osmundacetone is most preferably used. For example, the Osmunda japonica extract may be manufactured by selecting a proper extraction solvent and using an extraction method that is known in the art, such as acid/base extraction, hot-water extraction, room-temperature stirring extraction, cold extraction, reflux cooling extraction, ultrasonic extraction, autoclave extraction, low-temperature high-pressure extraction, enzyme treatment extraction, or solvent extraction.

As the extraction solvent, at least one solvent selected from the group consisting of water, ethanol, grain ethanol, methanol, propanol, isopropanol, butanol, acetone, ether, chloroform, ethyl acetate, methylene chloride, hexane, cyclohexane, petroleum ether, diethyl ether, and benzene may be used.

The Osmunda japonica extract according to the present invention may be an extract that is primarily extracted by the solvent extraction method, or may be obtained by mixing a primary extract and an extract resulting from re-extraction of an extract residue after primary extraction, in order to increase efficiency of extraction. In order to remove impurities and increase the concentration of an active ingredient, the Osmunda japonica extract may be obtained by further carrying out various purifying or filtering processes, such as separation by chromatography, fraction, diatom filtration, and ultrafiltration (membrane separation), according to methods known in the art. The final extract may be concentrated by using known concentration methods and concentration apparatuses, such as precipitation concentration, evaporation concentration, azeotropic concentration, vacuum concentration, distillation concentration, centrifugation, and reverse osmosis, and may be prepared in a powder form by removing solvents through freeze drying, spray drying, hot-air drying, and the like, followed by solidification.

In order to increase the content of osmundacetone in the Osmunda japonica extract, a hot-water extract is preferably prepared from Osmunda japonica, and the hot-water extract is again prepared into an extract and a fraction using an organic solvent, such as ethyl acetate.

Carriers that may be contained in the pharmaceutical composition containing the Osmunda japonica extract as an active ingredient, the formulation of the pharmaceutical composition, and the method of administration, such as the route of administration and the amount of administration, are as described above.

Furthermore, the present invention provides a food composition for alleviating a bone disease, the composition containing an Osmunda japonica extract as an active ingredient, wherein the bone disease is at least one selected from the group consisting of osteoporosis, rheumatoid arthritis, arthralgia, Paget's disease, bone metastatic cancer, and bone fractures.

The Osmunda japonica extract for preparing the food composition is as described above. Examples of the food composition and the contents thereof are also as described above.

Furthermore, the present invention provides a use of osmundacetone or a pharmaceutically acceptable salt thereof for the preparation of a preparation for preventing or treating a bone disease, wherein the bone disease is at least one selected from the group consisting of osteoporosis, rheumatoid arthritis, arthralgia, Paget's disease, bone metastatic cancer, and bone fractures.

As used herein, the term “pharmaceutically acceptable” refers to being physiologically acceptable, and not usually causing an allergic response or a similar response when administered to a human being. An acid addition salt formed by a pharmaceutically acceptable free acid is preferable as the salt. An inorganic acid and an organic acid may be used as the free acid. Examples of the organic acid include, but are not limited to, citric acid, acetic acid, lactic acid, tartaric acid, maleic acid, fumaric acid, formic acid, propionic acid, oxalic acid, tripleuroacetic acid, benzoic acid, gluconic acid, meta sulfonic acid, glycolic acid, succinic acid, 4-toluenesulfonic acid, glutamic acid, and aspartic acid. Examples of the inorganic acid include, but are not limited to, hydrochloric acid, bromic acid, sulfuric acid, and phosphoric acid.

As for a dose of the preparation for treatment of the present invention, an appropriate effective amount thereof may be determined according to the foregoing particular uses by a person skilled in the art considering various factors, such as the route of administration, the time of administration, the number of times of treatment, the period of treatment, and the age, weight, health condition, sex, severity of disease, susceptibility to drugs, diet, and excretion rate of a subject in need of treatment. The term “effective amount” refers to the amount sufficient to show effects of alleviating, treating, or preventing the bone disease when administered to a subject. The term “subject” may be an animal, preferably a mammal, and more preferably, an animal including a human being, and may be cells, a tissue, an organ, or the like, derived from an animal. The subject may be a bone disease patient in need of treatment.

The administration may be performed once a day or divided into several times. The preparation for treatment of the present invention may be administered alone or co-administered with another therapeutic agent known to have effects on the prevention or treatment of a bone disease. In a case of the co-administration, the pharmaceutical composition and another therapeutic agent may be administered sequentially or simultaneously. The dose of the preparation for treatment of the present invention when administered alone or in combination is preferably such that the maximum effect can be obtained in a minimal amount without side effects, and such an amount can be easily determined by a person skilled in the art.

The osmundacetone is characterized by being isolated from a plant in the family Osmundaceae, and a method for the isolation is as described above.

Furthermore, the present invention provides a method for treating a bone disease, the method comprising administering an effective amount of osmundacetone or a pharmaceutically acceptable salt thereof to a subject in need thereof, wherein the bone disease is at least one selected from the group consisting of osteoporosis, rheumatoid arthritis, arthralgia, Paget's disease, bone metastatic cancer, and bone fractures.

As used herein, the term “treatment” means a clinical procedure intended to alter a natural course of an individual or cell to be treated, and the treatment may also be performed for the prevention of clinical pathology. Preferable effects of the treatment include suppressing occurrence or recurrence of disease, relieving symptoms, reducing direct or indirect pathological consequences of disease, reducing disease progression rates, improving, bettering, or relieving disease conditions, or improving prognosis. As used herein, the term “prevention” refers to all actions that suppress the occurrence of diseases or delays the progress of disease.

Furthermore, the present invention provides a use of an Osmunda japonica extract for the preparation of an agent for preventing or treating a bone disease, wherein the bone disease is at least one selected from the group consisting of osteoporosis, rheumatoid arthritis, arthralgia, Paget's disease, bone metastatic cancer, and bone fractures.

The Osmunda japonica extract can be used without limitation as long as the extract is known to be obtained by a natural product extraction method. Especially, an extraction method capable of preparing an extract containing osmundacetone is most preferably used. For example, the Osmunda japonica extract may be manufactured by selecting a proper extraction solvent and using an extraction method that is known in the art, such as acid/base extraction, hot-water extraction, room-temperature stirring extraction, cold extraction, reflux cooling extraction, ultrasonic extraction, autoclave extraction, low-temperature high-pressure extraction, enzyme treatment extraction, or solvent extraction.

As the extraction solvent, at least one solvent selected from the group consisting of water, ethanol, grain ethanol, methanol, propanol, isopropanol, butanol, acetone, ether, chloroform, ethyl acetate, methylene chloride, hexane, cyclohexane, petroleum ether, diethyl ether, and benzene may be used.

The Osmunda japonica extract according to the present invention may be an extract that is primarily extracted by the solvent extraction method, or may be obtained by mixing a primary extract and an extract resulting from re-extraction of an extract residue after primary extraction, in order to increase efficiency of extraction. In order to remove impurities and increase the concentration of an active ingredient, the Osmunda japonica extract may be obtained by further carrying out various purifying or filtering processes, such as separation by chromatography, fraction, diatom filtration, and ultrafiltration (membrane separation), according to methods known in the art. The final extract may be concentrated by using known concentration methods and concentration apparatuses, such as precipitation concentration, evaporation concentration, azeotropic concentration, vacuum concentration, distillation concentration, centrifugation, and reverse osmosis, and may be prepared in a powder form by removing solvents through freeze drying, spray drying, hot-air drying, and the like, followed by solidification.

In order to increase the content of osmundacetone in the Osmunda japonica extract, a hot-water extract is preferably prepared from Osmunda japonica, and the hot-water extract is again prepared into an extract and a fraction using an organic solvent, such as ethyl acetate.

Furthermore, the present invention provides a method for treating a bone disease in a subject, the method comprising administering an effective amount of an Osmunda japonica extract to a subject in need thereof, wherein the bone disease is at least one selected from the group consisting of osteoporosis, rheumatoid arthritis, arthralgia, Paget's disease, bone metastatic cancer, and bone fractures.

The effective amount of the Osmunda japonica extract and the administration method, such as the route of administration and the amount of administration, are as described above.

### Advantageous Effects

Therefore, the present invention provides a composition for the prevention, alleviation, or treatment of at least one bone disease selected from the group consisting of osteoporosis, rheumatoid arthritis, arthralgia, Paget's disease, bone metastatic cancer, and bone fractures, the composition comprising, as an active ingredient, osmundacetone or a derivative thereof, or an Osmunda japonica extract comprising the same. The composition according to the present invention has very low toxicity, and shows a strong inhibitory effect on the proliferation and differentiation of osteoclasts causing bone loss, and simultaneously shows an effect of activating the differentiation of osteoblasts.

### BRIEF DESCRIPTION OF THE DRAWINGS

FIG. 1 shows HPLC results of water (hot water) or ethyl acetate (EA) extract of Osmunda japonica in order to isolate and identify a substance having osteoclast differentiation inhibitory activity contained in Osmunda japonica (detection wavelength: 280 nm). EA-2 represents the second fraction among EA extracts separated as seven fractions. The peak of osmundacetone was marked by a black arrow. The peaks marked with red ellipses indicate the same peak position observed during a purification process.

FIG. 2 shows TRAP assay results using mouse bone marrow cells in order to investigate the activity of Osmunda japonica extracts, a fraction thereof, and osmundacetone to inhibit the proliferation and differentiation of osteoclasts.

FIG. 3 shows TRAP assay results using mouse bone marrow cells to investigate the osteoclast proliferation and inhibition inhibitory activity of 1, 4, 7, and 10 μM osmundacetone commercially purchased (Alfa Aesar, Thermo Fisher Scientific) in order to obtain the osteoclast differentiation inhibitory ability, IC₅₀, of osmundacetone.

FIG. 4A shows the results of confirming IC₅₀ of osmundacetone and the known drug Fosamax after bone marrow cells were treated with various concentrations of osmundacetone and Fosamax to induce differentiation thereof, in order to compare the osteoclast differentiation inhibition effect between osmundacetone and Fosamax. FIG. 4B shows the results of confirming the ability of osmundacetone to inhibit osteoclast differentiation and activate osteoblast differentiation simultaneously while osteoclast precursor cells and osteoblast precursor cells were co-incubated.

FIG. 5 shows the results of confirming the expression of OCN by performing western blot after the treatment with osmundacetone, in order to investigate the effect of osmundacetone on the OCN production of osteoblasts.

FIG. 6 shows the results of confirming the expression of RUNX2 by performing western blot after the treatment with osmundacetone, in order to investigate the effect of osmundacetone on the expression of RUNX2.

### MODE FOR CARRYING OUT THE INVENTION

Hereinafter, the present invention will be described in detail.

However, the following examples are merely for illustrating the present invention and are not intended to limit the scope of the present invention.

### <Example 1> Experimental Methods

1. Preparation of Osmunda japonica Extracts and Fractions and Isolation and Identification of Compound

Osmunda japonica (or Osmundae Rhizoma) was extracted with hot water and ethyl acetate sequentially, and then an ethyl acetate extract (EA extract) was separated into seven fractions through HPLC.

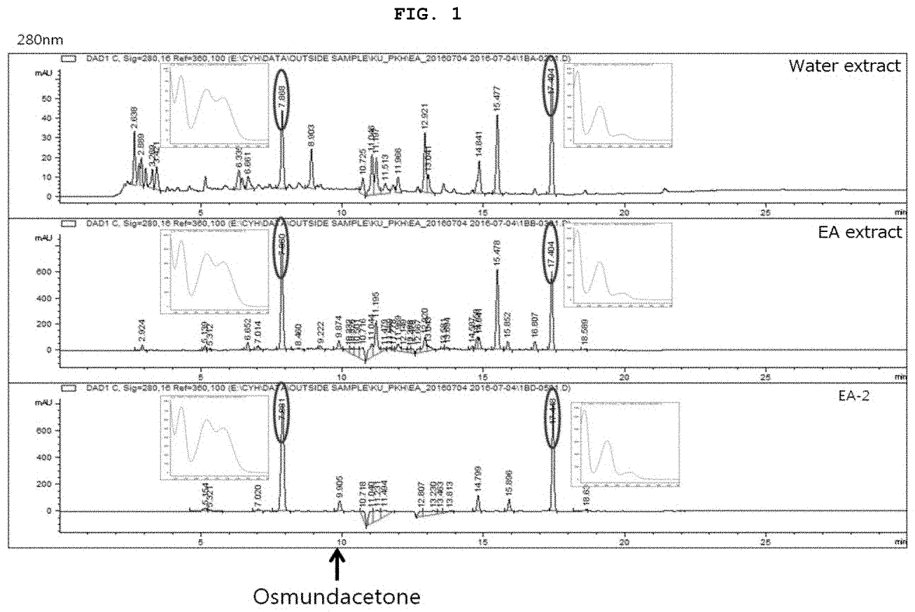

Specifically, as the Osmunda japonica extract, a hot-water extract obtained by clearly washing 200-250 g of Osmunda japonica collected in Gangwon-do, Korea, placing the washed Osmunda japonica in a steaming container (OSK-2002, Doctor Red Ginseng, Well Sosana™, Daewoong Pharmaceutical Inc.), adding 1.5 L of water, steaming for 24 hours, further adding 3.5 L of water, aging for 72 hours, and refrigerating, was used. The same volume of ethyl acetate (EA) was added to the hot-water extract, followed by well mixing, and then the EA layer was dried using a rotary evaporator, and then used as an EA extract. The EA extract was dissolved in a minimum amount of DMSO, and then diluted with water, and here, as for a dilution factor, the EA extract was diluted to 70% of the original volume of the hot-water extract assuming a yield of approximately 70%. The EA extract was separated into seven fractions through HPLC. The second fraction was dried using a rotary evaporator, and then used as EA-2 extract. It was observed that among the seven fractions, the second fraction (EA-2) had osteoclast differentiation inhibitory activity (see FIG. 1, <Example 2>).

In order to identify a substance having osteoclast differentiation inhibitory activity, nine single compounds were isolated from the EA-2 fraction, followed by purification, and then the kind and chemical structure of each compound were established through nuclear magnetic resonance (NMR) and mass spectrometry (MS). The conditions of HPLC for isolating the single compounds from the EA-2 fraction are shown in Table 1. The substance showing osteoclast differentiation inhibitory activity among the isolated compounds was confirmed to be osmundacetone (see <Example 2>). Specific NMR and MS results of the osmundacetone identified from the Osmunda japonica extract are as follows:

¹H-NMR (700 MHz, methanol-d4) δ7.52 (1H, d, J=16.1, H-7), 7.08 (1H, d, J=2.1 Hz, H-2), 6.99 (1H, dd, J=7.7, 2.1 Hz, H-6), 6.79 (1H, d, J=7.7 Hz, H-5), 6.55 (1H, d, J=16.1 Hz, H-8), 2.34 (3H, s, H-10); ¹³C-NMR (175 MHz, methanol-d4) δ 201.7 (C-9), 150.1 (C-4), 147.1 (C-3), 147.0 (C-7), 127.9 (C-1), 124.9 (C-8), 123.7 (C-6), 116.7 (C-5), 115.4 (C-2), 27.2 (C-10); ESI-MS (negative mode) m/z 177 [MH]; ESIMS (positive mode) m/z 179 [M+H]+, 201 [M+Na]⁺.

TABLE 1
EA-2 fraction HPLC separation method

| | |
|---|---|
| HPLC | Agilent 1200 |
| Column | Kromasil 100-5-C18 (4.6*250 mm) |
| Detector | UV (210 nm, 254 nm, 280 nm) |
| Flow | 1 ml/min |
| Oven | 30° C. |
| Injection | 10 μl |

| Mobile phase | A (0.05% TFA in H₂O) % | B (MeOH) % |
|---|---|---|
| 0 min | 80 | 20 |
| 20 min | 0 | 100 |
| 30 min | 0 | 100 |

2. Cell Culture

MC3T3-E1 subclone 4 murine osteoblastic cells, (# CRL-2593TM, ATCC) were incubated using α-MEM (Minimum Essential Medium Eagle-Alpha Modification, # LM00853, WELGENE, Seoul, Korea) in the conditions of 37° C. and 5% CO₂.

HaCaT human epidermal keratinocytes, # ATCC PCS-200-011) were incubated using Dulbecco's modified Eagle's medium (DMEM, # DMEM-HPA, Capricorn Scientific, Ebsdorfergrund, Germany) in the conditions of 37° C. and 5% CO₂. NIH3T3 mouse embryo fibroblasts (# KCLB 21658), RAW264.7 murine macrophages (preosteoclast cell line, # KCLB 40071), HCT116 human colon cancer cells (# KCLB 10247), PC3 human prostate adenocarcinoma cells (# KCLB 21435), HT1080 human fibrosarcoma cells (# KCLB 1121), and B16F10 mouse malignant melanoma cells (# KCLB 80008) were purchased from the Korean Cell Line Bank, and incubated using DMEM in the conditions of 37° C. and 5% C02.

AGS human stomach adenocarcinoma cells (# KCLB 21739), A549 human lung carcinoma cells (# KCLB 10185), Caki-1 human kidney carcinoma cells (# KCLB 30046), T24 human bladder carcinoma cells (# KCLB 30004), TC-1 P3 HPV-16E7-expressing mouse pulmonary epithelial cells, MHC class I (provided by Tae-Woo Kim, a professor of Korea University) were incubated using RPMI (Rosewell Park Memorial Institute) 1640 (# RPMI-A, Capricorn Scientific, Ebsdorfergrund, Germany) in the conditions of 37° C. and 5% CO₂.

Human adipose-derived mesenchymal stem cells, ADMSCs (CEFO Bio, Seoul, Korea), were incubated using CB-ADMSC-GM (CEFO Bio, Seoul, Korea) in the conditions of 37° C. and 5% CO₂.

3. Primary Culture of Osteoclasts

Bone marrow cells were collected from the femur and shin bones of 5-8 week old male C57BL/6 mice. Muscles were removed from the bones, and stored in cold phosphate buffered saline (PBS; # CAP08-050, GenDEPOT, Katy, Tex., USA).

Both ends of each bone were cut, and the bone marrow was flushed with a flushing medium (serum-free α-MEM, 2 mM ethylenediaminetetraacetic acid) kept cool, using a 25 G needle.

Bone marrow cells were collected by centrifugation at 3,000 rpm for 3 min using Labogene 1248R (Labogene, Lynge, Denmark), and then resuspended in a wash medium. Thereafter, 8 mL of the collected bone marrow cells were overlaid on 6 mL of a lymphocyte isolation medium (LSM; #50494, MP Biomedicals, Santa Ana, Calif., USA), followed by centrifugation at 1,600 rpm for 20 minutes, thereby isolating mononuclear cells.

The mononuclear cell bands were collected from the media interface, and incubated using complete α-MEM (containing 10% fatal bovine serum (FBS; Capricorn Scientific, Ebsdorfergrund, Germany) and 1% (v/v) antibiotic (including 100 U/mL penicillin G and 100 mg/mL streptomycin)) in the conditions of 37° C. and 5% CO₂ while the medium was exchanged every three days.

In order to promote the differentiation of osteoclasts, 1×10⁵ cells were incubated in 0.5 mL of the medium per well in a 48-well plate, in the presence of osteoclast differentiation factors, M-CSF (60 ng/mL) and RANKL (150 ng/mL), purchased from PeproTec (Seoul, Korea). As known, macrophages/monocytes derived from one bone marrow differentiated into mature multinucleated osteoclasts after 6 days (Gurt et al., 2015).

4. Co-Culture of Osteoclasts and Osteoblasts

1×10⁵ C57BL/6 mouse bone marrow mononuclear cells per well of a 48-well plate were prepared in the same manner as described above, and co-incubated together with 1.5×10⁴ MC3T3-E1 murine osteoblasts, using 500 μL of α-MEM containing 10% FBS and 1% antibiotic (100 U/mL penicillin G and 100 mg/mL streptomycin) in the conditions of 37° C. and 5% CO₂ while the medium was exchanged every three days.

Positive control cells were co-incubated together with osteoclast differentiation factors M-CSF (60 ng/mL) and RANKL (150 ng/mL), and osteoblast differentiation factors ascorbic acid (50 μg/mL) and 10 mM R-glycerophosphate.

Negative control cells were incubated by adding only M-CSF as a differentiation factor. In order to examine effects of osmundacetone on the differentiation of osteoclasts and osteoblasts, osmundacetone were administered at a final concentration of 10 μM into the positive control cells on day 1 after the cells were dispensed on the plate.

In the positive control group treated with osmundacetone, osteoclasts completely disappeared 6-7 days after cell dispensing, and thus while only osteoblast differentiation factors were administered, the cells were incubated from 8 days to 21 days after cell dispensing.

5. Analysis of Bone Resorption

The quantitative measurement of in vitro osteoclast-mediated degradation of human bone collagen was carried out using the OsteoLyse™ Assay Kit (Lonza Walkersville, Inc. Walkersville, Md., USA) according to manufacturer's instructions.

This assay allows a direct measurement of the release of matrix metalloproteinase into the osteoclast resorption lacuna (Delaisse et al., 2003).

Briefly, 2×10⁴ mouse bone marrow cells prepared above were dispensed in 96-well OsteoLyse™ cell culture plate coated with europium-conjugated collagen.

The cells were incubated in 0.1 mL of complete α-MEM per well in the presence of M-CSF (60 ng/mL) and RANKL (150 ng/mL) in the conditions of 37° C. and 5% CO₂ for 6 days. Thereafter, the medium was exchanged on day 3 of the dispensing.

In order to measure the ability to inhibit the mature osteoclastic functions, the medium was exchanged on day 6, and osmundacetone was added at an IC₅₀ concentration obtained from TRAP analysis.

After the mature osteoclasts were treated with osmundacetone for 3 days, 10 μL of a supernatant of the cell culture was taken, and placed in a second 96-well analysis plate containing fluorophore-Releasing reagent. The degraded collagen was measured using a time-resolved fluorescence fluorimeter, Wallac Victor (Perkin Elmer, Waltham, Mass., USA). Here, the measurement was carried out using excitation at 340 nm and emission at 615 nm for a time interval of 400 μs after an initial delay of 400 μs.

The bone resorption rate (%) was obtained by calculating the proportion of the amount of bone resorption by the presence of osmundacetone compared with the non-treated control group, and was normalized by cellular DNA.

6. Western Blot

In order to analyze the expression of osteoblast differentiation markers during a differentiation procedure, 3×10⁵ MC3T3-E1 cells were dispensed in a 100 mm culture plate together with 10 mL of complete α-MEM containing ascorbic acid (50 μg/mL) and 10 mM R-glycerophosphate per well. Here, 50 μM osmundacetone was added or not added, and the cells were incubated at 37 [symbol] É in a 5% CO₂ incubator for 21 days while the medium was exchanged every 3-4 days.

The negative control cells were cultured without differentiation factors, and the osteoblasts and a culture thereof were dispensed, and then collected on day 7, 14, or 21.

The cells were lysed using RIPA buffer, and then the expression of runt-related transcription factor 2 (RUNX2) was analyzed. Also for analysis of OCN secretion, the culture was collected, and normalized by cellular DNA.

Proteins were analyzed using SDS-PAGE on 12.5% polyvinylidene-Tris gels, and transferred on polyvinylidene difluoride (PVDF) membrane through electrophoresis.

The membrane was blocked using non-fat milk, and examined using anti-RUNX2 (D1H7) rabbit monoclonal antibody (#8486, Cell Signaling Technology, MA, USA) or anti-OCN (FL-95) antibody (# sc-30045, Santa Cruz Biotechnology, TX, USA). Anti-actin antibody (# M177-3, MEDICAL & BIOLOGICAL LABORATORIES CO., LTD., Nagoya, Aichi, Japan) was used as an internal reference.

Protein bands on the blot were visualized using an enhanced chemiluminescent detection kit (# EBP-1073, PicoEPD Western Reagent, ELPIS-BIOTECH, Daejeon, Korea).

7. Measurement of ALP Activity

ALP activity was assessed using Quantichrom ALP Assay Kit (Bioassay Systems, Hayward, Calif., USA) according to manufacturer's instructions.

Briefly, 3×10³ MC3T3-E1 cells were dispensed in a 96-well plate together with 10 μL of complete α-MEM containing ascorbic acid (50 μg/mL) and 10 mM β-glycerophosphate per well. Here, osmundacetone (10 μM and 50 μM) was added or not added, and the medium was exchanged every 3-4 days.

On day 14 of the incubation, the colorimetric change due to ALP activity in the cell fraction was measured using a spectrophotometer plate reader (Molecular Devices, Sunnyvale, Calif., USA) at 405 nm (Kim et al., 2016). The % activation of ALP activity was shown by comparing ALP activity of cells treated with an experimental compound and ALP activity of the non-treated control cells.

8. TRAP Assay (Measurement of Osteoclast Proliferation and Differentiation Inhibitory Activity)

1) Culture of Bone Marrow Cells

The tibia and femur of 6-8 week old male C57BL/6 mice were aseptically resected, and bone marrow cells were aseptically collected using a syringe (21 G, Korea Green Cross). The bone marrow cells were floated in 500 μL of α-MEM medium (Gibco BRL Co.) containing sodium bicarbonate (2.0 g/L), streptomycin (100 mg/L), and penicillin (100,000 unit/mL), dispensed in a 48-well plate, and assayed in triplicate. Mononuclear cells as precursor cells of osteoclasts were treated with RANKL and M-CSF as differentiation promoting factors, and thus differentiated into osteoclasts within 5-7 days.

2) Measurement of Inhibition of Osteoclast Differentiation

2-1) Sample preparation: The hot-water extract, EA extract, or fraction of Osmunda japonica were prepared by the same methods as in section 1 of <Example 1>. The EA-2 extract was dissolved in a minimum amount of DMSO, and then diluted with water to 70% of the original volume of the hot-water extract assuming that the yield of EA-2 extraction was approximately 70%. 2-2) Sample administration: The sample was continuously administered to a medium at 1:20 (v/v; 25 μL of the sample per 500 μL of the medium) from day 1 of incubation of bone marrow cells, while the medium was exchanged every 2-3 days. 2-3) Measurement of osteoclast differentiation Osteoclasts were defined by TRAP-positive multinucleated cells stained with TRAP. As for TRAP stain solution, 5 mg of naphthol AS-MS phosphate (Sigma N-4875) as a base and 25 mg of Fast Red Violet LB salt as a color developing reagent were dissolved in about 0.5 mL of N,N-dimethylformamide, and then mixed with 0.1N NaHCO₃ buffer solution (50 mL) containing 50 mM tartaric acid. The reaction reagent was stored in a refrigerator before use.

After bone marrow cells were incubated in a medium containing differentiation promoting factors for 7 days, the medium was removed, and the cells were washed with PBS, and then immobilized with PBS containing 10% formalin for 2-5 minutes. Thereafter, the cells were immobilized with a 1:1 mixture solution of ethanol and acetone, followed by drying. The immobilized cells were treated with the TRAP stain solution for 15 minutes, and washed with PBS, and then the degree of cell staining was observed by a microscope.

In the microscope field of view, cells having two or more nuclei in the TRAP-positive cells were determined to be osteoclasts, and the number of cells was measured. The osteoclast differentiation inhibitory effect of the Osmunda japonica extract was calculated by IC₅₀ as the 50% inhibitory concentration compared with the control group.

9. TRAP Assay (Determination of Osteoclast Proliferation and Differentiation Inhibitory Activity IC₅₀)

1) Measurement of Osteoclast Differentiation

1-1) Sample preparation: Osmundacetone was purchased from the Alfa Aesar, Thermo Fisher Scientific, and a minimal amount thereof was dissolved in dimethylsulfoxide (DMSA). Fosamax was purchased from Cayman (Ann Arbor, Mich., USA), and a minimal amount thereof was dissolved in sterile distilled water.

1-2) Sample administration: Osmundacetone and Fosamax were continuously administered to a medium at 1:20 (v/v; 25 μL of the sample per 500 μL of the medium) from day 1 of incubation of bone marrow cells such that the final concentrations were 1, 4, 7, and 10 μM, respectively, while the medium was exchanged every 2-3 days.

1-3) Measurement of osteoclast differentiation: The measurement was carried out by the same method as in section 8 in <Example 1>.

In the microscope field of view, cells having two or more nuclei in the TRAP-positive cells were determined to be osteoclasts, and the number of cells was measured. The osteoclast differentiation inhibitory effect of osmundacetone was calculated by IC₅₀ as the 50% inhibitory concentration compared with the control group.

10. Investigation of Cytotoxicity

The compound isolated from Osmunda japonica, prepared in section 1 of <Example 1>, was investigated for cytotoxicity using MTT assay.

MTT assay was as follows.

Cells were incubated at 1×10³ cells/well in a 96-well plate containing DMEM with 10% fetal bovine serum (FBS) at 5% CO₂ and 37° C., and then osmundacetone was added to the cell medium, followed by incubation for 24 hours. Thereafter, 100 μL of MTT (0.5 mg/ml PBS) was administered, followed by incubation for 2 hours. Thereafter, the medium was removed from each well, and 100 μL of DMSO was added. After incubation for 10 minutes, the absorbance was measured using a microplate reader (SPCTRA MAX 340PC, Molecular Devices, USA) at 570 nm. The absorbance is an indicator showing the number of living cells, and calculated by the following equation. The reproducibility thereof was validated by three experiments.

Cell proliferation (%)=OD₅₅₀(sample)/OD₅₅₀(control)

### <Example 2> Results

1. Confirmation of Osteoclast Proliferation and Differentiation Inhibitory Activity

The extract, fraction, and isolated compound of Osmunda japonica, prepared in <Example 1>, were investigated for osteoclast proliferation and differentiation inhibitory activity using the tartrate-resistant acid phosphatase (TRAP) assay, which is an osteoclast-specific staining method.

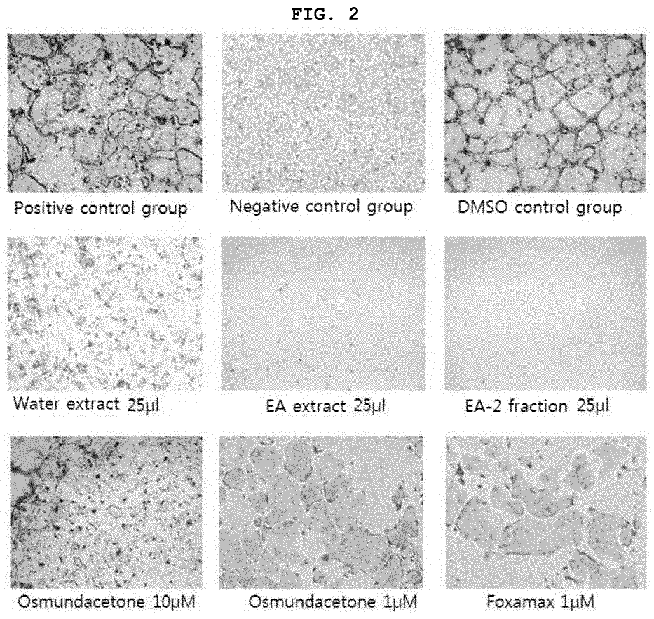

As can be confirmed from FIG. 2, giant osteoclasts were normally formed in the bone marrow cells treated with DMSO, like in the positive control group (a group with only differentiation promoting factors added to a culture medium without an Osmunda japonica extract). In contrast, the formation of giant osteoclasts as multinucleated cells was significantly inhibited in the group treated with the same volume of the water extract, EA extract, EA-2 fraction, and osmundacetone (10M) of Osmunda japonica similar to the negative control group (a group with neither differentiation promoting factors nor Osmunda japonica extract and added to a culture medium), and also, in addition to the results like in the negative control group, the proliferation of osteoclast precursor cells was significantly inhibited, leading to great inhibitory effects on both differentiation and proliferation of osteoclasts. Especially, when the cells were treated with 10 μM osmundacetone, the differentiation of bone marrow mononuclear cells into multinucleated cells, osteoclasts, through proliferation and fusion was 95% or more, almost complexly inhibited. The group treated with 1 μM osmundacetone showed the formation of giant osteoclasts, the number of which was smaller compared with the positive control group. The group treated with 1 μM Fosamax also showed the formation of giant osteoclasts, the number of which was smaller compared with the positive control group, and showed an osteoclast density similar to that in the group treated with 1 μM osmundacetone.

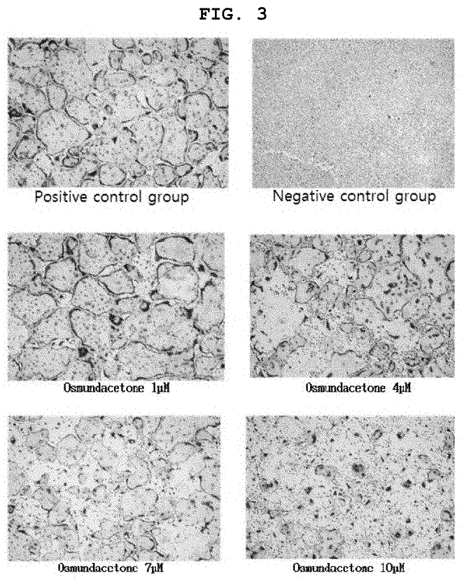

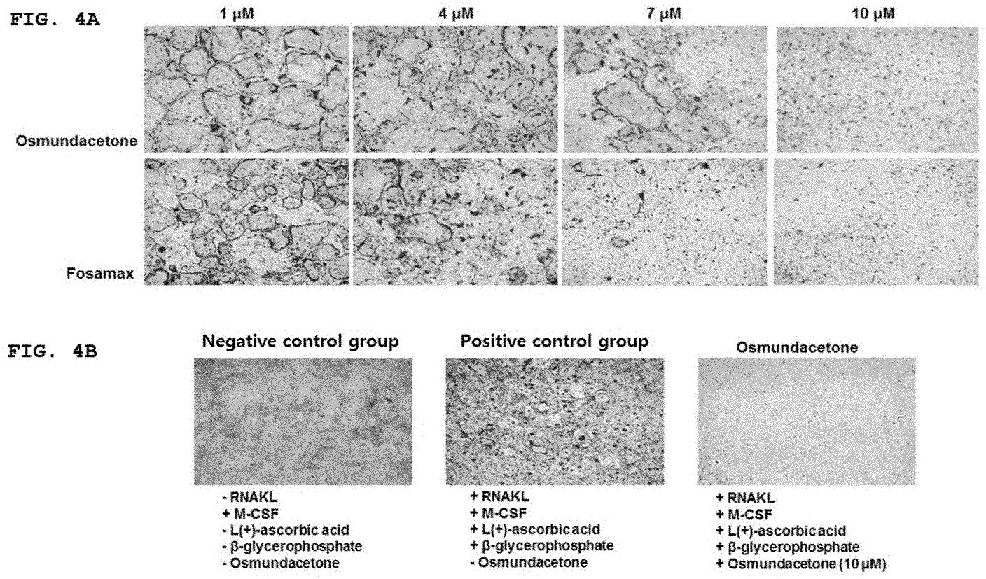

Also, the osteoclast proliferation and differentiation inhibitory activity IC₅₀ was obtained. As can be confirmed from FIG. 3, the differentiation of bone marrow mononuclear cells into multinucleated cells, osteoclasts, through proliferation and fusion was almost completely inhibited when the cells were treated with 10 μM osmundacetone (Alfa Aesar, Thermo Fisher Scientific), and was about 3-40% inhibited when the cells were treated with 7 μM osmundacetone (IC₅₀=≤8 μM). The known osteoporosis medicine, Fosamax (alendronate), which was used as a positive control, had an IC₅₀ value of ≤4 μM (FIG. 4A).

In order to investigate whether or not osmundacetone inhibited the bone resorption function of completely differentiated mature osteoclasts, the bone resorption assay using mature osteoclasts was carried out in the presence of osmundacetone at 8 μM, the IC₅₀ concentration.

Osmundacetone inhibited the bone resorption function of mature osteoclasts up to 58.7±13% that of non-treated osteoclasts at the IC₅₀ concentration on the basis of the method in section 5 of <Example 1>.

2. Confirmation of Effects of Osmundacetone on Activation of Osteoblasts and Inhibition of Osteoclast Differentiation

In order to investigate whether or not osmundacetone has ability to inhibit osteoclast differentiation and activate osteoblast differentiation simultaneously, bone marrow mononuclear cells collected from C57BL/6 mice and MC3T3-E1 cells as osteoblast precursor cells were co-incubated in a 48-well plate at cell concentrations of 1×10⁵ bone marrow cells and 3×10³ MC3T3-E1 cells per well. The bone marrow monocyte/macrophage lineage cells in the co-incubated bone marrow cells and osteoblast precursor cells differentiated into mature multinuclear osteoclasts in the presence of M-CSF and RANKL within 6-7 days.

The administration of 10 μM osmundacetone completely inhibited the proliferation and differentiation of osteoclasts (FIG. 4B, Os). Whereas the osteoblasts continuously proliferate and differentiate. The % activation values of ALP activity on days 7, 14, and 21 of co-incubation when 10 μM osmundacetone was administered were 104%, 111%, and 95%, respectively, compared with when osmundacetone was not administered, and these results were not greatly different from the ALP activation of osteoblasts incubated alone after the administration of 10 μM osmundacetone.

Osmundacetone showed similar inhibitory activities on osteoclast differentiation in the presence and absence of osteoblasts. On day 6 after the administration of 10 μM osmundacetone, mature osteoclasts completely disappeared while osteoblasts continuously proliferated. Therefore, as shown from the co-incubation of preosteoclasts and preosteoblasts, osmundacetone did not show inhibitory activity on the activation and proliferation of co-existing osteoblasts.

On the basis of the above results, it was confirmed that osmundacetone has ability to inhibit osteoclast differentiation and activate osteoblast differentiation simultaneously.

In contrast, in the absence of osmundacetone, both osteoclasts and osteoblasts continuously proliferated and differentiated in the presence of osteoclast and osteoblast differentiation factors 7 days after the co-incubation of preosteoclasts and preosteoblasts (FIG. 4B, Positive control group).

However, the size of differentiated osteoclasts were somewhat small when compared with the size of osteoclasts grown in the absence of osteoblasts, and the reason may be due to cell density increased due to co-existence of osteoclasts and osteoblasts.

The cells grown in the incubation of the negative control group were most likely to configure macrophage/mononuclear cell lineage cells since only M-CSF was used as a differentiation factor (FIG. 4B, the negative control group).

Overall, these results indicated that osmundacetone has ability to inhibit osteoclast differentiation and activate osteoblast differentiation independently and simultaneously.

3. Confirmation of Increasing Effects of Osmundacetone on ALP and OCN Production by Osteoblasts

In order to assess the ability of osmundacetone to induce bone formation, it was investigated whether or not osmundacetone increased the activity of alkaline phosphatase (ALP), which is a marker for initial/intermediate steps of osteoblast differentiation.

TABLE 2
% Activation of ALP activity of osmundacetone in MC3T3-E1

% Activation of ALP activity

| Compounds | 10 μM | 50 μM |
|---|---|---|
| Osmundacetone | 115 ± 9.4 | 279 ± 61* |
| Parathyroid hormone-related | 138 ± 19 | |
| peptide (1 μM) | | |

The above values are expressed by mean±s.d. of three independent experiments. * represents P<0.05. The parathyroid hormone-related peptide was used as a positive control.

As shown in Table 1 above, the ATP production in the osteoblast-like MC3T3-E1 cells treated with osmundacetone was significantly increased compared with ALP production in non-treated control cells.

Similar to the results shown in the previously known literature (Lyu et al., 2008), 50 μM osmundacetone stimulated ALP production by 279±61% (P<0.05).

In addition, osmundacetone showed a tendency to increase ALP production by MC3T3-E1 cells regardless of the inhibition of differentiation of co-existing osteoclasts. When osteoblast and osteoclast precursors were co-incubated with 10 μM osmundacetone and osteoclast and osteoblast differentiation factors, mature osteoclasts completely disappeared on day 6 (FIG. 4B, Os).

Thereafter, the cells treated with osmundacetone were continuously grown in the presence of osteoblast differentiation factors.

The % activation values of ALP activity of the cells treated with osmundacetone, on days 7, 14, and 21 after the co-incubation of osteoblast and osteoclast precursor cells, were 104%, 111%, and 95%, respectively, compared with the non-treated control cells, and these results were almost similar to the % activation value obtained in the absence of co-existing osteoclasts (Table 1).

Therefore, the results showed that osmundacetone promoted osteoblast differentiation and, simultaneously, maintained ability to inhibit osteoclast differentiation.

It was reported that OCN, which is a main noncollagenous matrix protein, showed the most increased expression only at or near the time of mineralization, that is, about 21 days after the induction of MC3T3-E1 cell differentiation (Young et al., 1992).

In order to investigate the effect of osmundacetone on OCN production by osteoclasts, MC3T3-E1 cells were incubated in the presence of 50 μM osmundacetone.

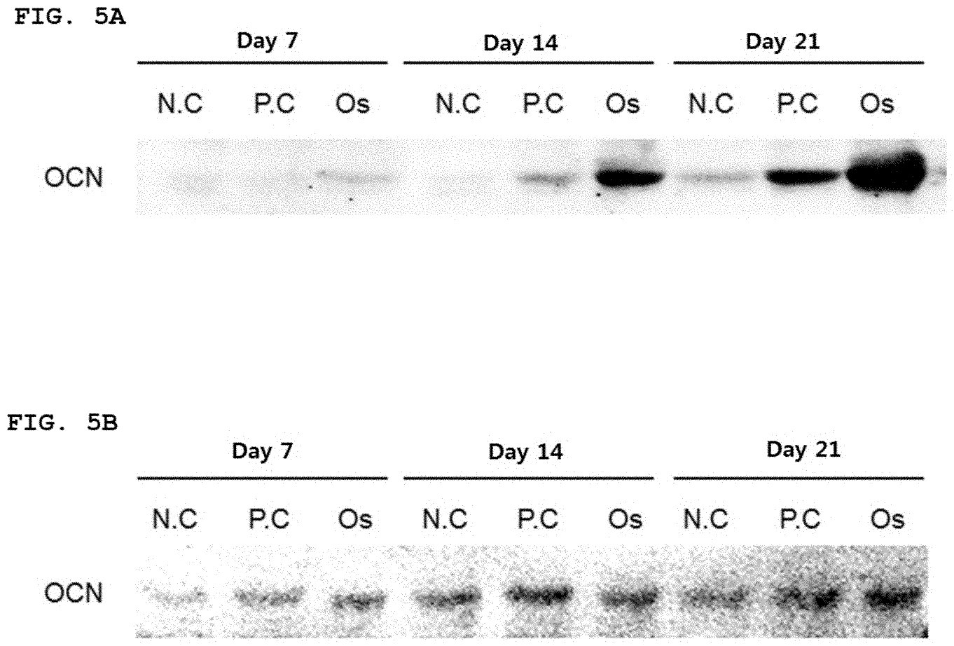

According to western blot assay, the levels of the OCN production by osteoblasts days 14 and 21 after the administration of osmundacetone were increased by 2.9 times and 1.2 times compared with the non-treated positive control cells, respectively (FIG. 5A, Os).

These results confirmed that osmundacetone initially induced a remarkable increase in OCN production by osteoblasts.

Even in the co-incubation of preosteoblasts and preosteoclasts, osmundacetone maintained ability to initially increase OCN production and inhibit osteoclast differentiation (FIG. 5B, Os).

The positive control osteoblasts grown in the presence of osteoblast and osteoclast differentiation factors in the environment of co-incubation without the administration of osmundacetone initially induced and increased the OCN production to a similar level to the osteoblasts in the environment of co-incubation with osmundacetone treatment, from day 7 to day 21 after cell dispensing (FIG. 5B, P.C). The reason is thought that the increase of OCN production was initially induced by the interaction with osteoclasts.

Interestingly, the negative control preosteoblasts grown without osteoblast differentiation factors, compared with osteoblasts grown without osteoclasts, showed a significant increase in OCN production to a level in the positive control cells in the presence of osteoclasts on days 14 and 21.

These results suggested that in the absence of osteoblast differentiation factors, the interaction with bone marrow mononuclear cells or mature osteoclasts may contribute to an increase in OCN production in osteoblasts.

Therefore, it was confirmed that osmundacetone inhibited osteoclast differentiation and maintained the ability to increase ALP and OCN expression in osteoblasts.

4. RUNX2 Expression Increasing Effect of Osmundacetone

Then, it was investigated whether or not osmundacetone increased the expression of RUNX2, which is an initial differentiation marker of osteoblasts. It is known that RUNX2 is a transcriptional factor to increase bone formation by stimulating the transcription of ALP and OCN in osteoblasts (Phimphlai et al., 2006).

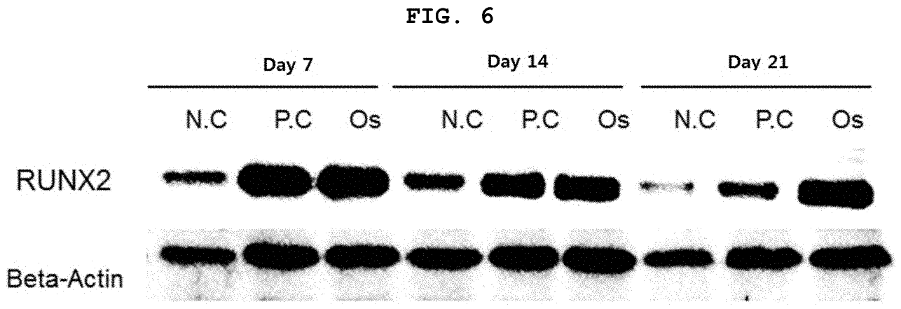

The expression levels of RUNX2 in MC3T3-E1 cells administered with osmundacetone on 7, 14, and 21 after the administration were increased by 1.1 times, 1.1 times, and 2.1 times compared with the non-treated positive control group (FIG. 6).

The RUNX2 expression in osteoblasts treated with osmundacetone was not significantly reduced until day 21 after cell dispensing, whereas the RUNX2 expression in non-treated osteoblasts on day 21 was reduced to almost half of the maximum expression level on day 14.

These results confirmed that osmundacetone had ability to extend the expression period of RUNX2 in osteoblasts.

5. Cancer Cell-Specific Cytotoxicity of Osmundacetone

Osmundacetone showed specific toxicity with a higher selectivity index for cancer cells than non-cancer cells.

Compounds having an IC₅₀ of less than 100 μM may be considered to be active in cell death/anti-proliferative activity (Boyd, 2003).

Therefore, referring to Table 3 below, osmundacetone did not show cell cytotoxicity on normal cell lines.

TABLE 3

| | LD₅₀ (μM) Based on MIT Assay | | | | | | | | | | | | | |
|---|---|---|---|---|---|---|---|---|---|---|---|---|---|---|
| | Normal cell | | | | Cancer cell | | | | | | | | | |
| Compounds | [symbol] | [symbol] | [symbol] | [symbol] | [symbol] GS | [symbol] 49 | [symbol] 62 | [symbol] 116 | [symbol] 3 | Cak-1 | [symbol] | [symbol] 1060 | [symbol] 10 | $C-1 P3 |
| Osmundacetone | [symbol] ± | [symbol] ± [symbol] | [symbol] ± [symbol] | >5,000 | [symbol] ± [symbol] | >5,000 | >5,000 | [symbol] ± [symbol] | [symbol] ± [symbol] | [symbol] ± [symbol] | [symbol] ± [symbol] | >5,000 | [symbol] ± [symbol] | [symbol] ± [symbol] |
| [symbol] | 1.10 | | | >5,000 | | | | | | | | | >5,000 | |

[symbol]

indicates data missing or illegible when filed

The above values are expressed by average±standard deviation of three independent experiments. Fosamax was used as a reference compound.

The cells used in the present experiments were as follows.

HaCaT, human epidermal keratinocytes; ADMSC, human Adipose-derived mesenchymal stem cells (CEFO, Korea); RAW264.7, mouse macrophage cell line (preosteoclasts); NIH3T3, mouse embryo fibroblasts; AGS, human stomach adenocarcinoma; A549, human lung carcinoma; HepG2, human liver hepatoblastoma; HCT116, human colon carcinoma; PC3, human prostate adenocarcinoma; Caki-1, human kidney carcinoma; T24, human bladder carcinoma; HT1080, human fibrosarcoma; B16F10, mouse melanoma; TC-1 P3, HPV-16 E7-expressing mouse lung epithelial cells (-MHC class I)

Especially, according to MTT assay, osmundacetone showed IC₅₀ values of 2,760±220, 3510±110, and >5,000 μM for ADMSC human Adipose-derived mesenchymal stem cells, HaCaT human epidermal keratinocytes, and NIH3T3 mouse embryo fibroblasts, respectively, indicating slight toxicity.

Meanwhile, osmundacetone showed an LD₅₀ of 507±98 μM for RAW264.7 mouse macrophage cell line, indicating relatively significant cytotoxicity. Therefore, these results suggested that osmundacetone may suppress the viability of some phagocytes at high concentrations.

Interestingly, according to MTT assay, osmundacetone showed low LD₅₀ of 59.9±6.1, 65.5±8.7, and 75.8±9.2 μM for cancer cell lines including AGS human stomach adenocarcinoma, PC3 human prostate adenocarcinoma, and B16F10 mouse melanoma, respectively, indicating moderate cytotoxicity activity.

Especially, the cancer selectivity index in cell cytotoxicity of osmundacetone was 45-60, very high in killing human cancer cell lines (AGS, PC3) compared with human normal cell lines (HaCaT, ADMSC).

In conclusion, the IC₅₀ of osmundacetone for inhibition of osteoclast inhibition was 8 μM, the concentration of osmundacetone to activate osteoblasts by 280% was 50 μM, and the LD₅₀ value of osmundacetone for several cancer cell lines was 50-70 μM, whereas the LD₅₀ of osmundacetone for normal cell lines was in a range of 2,500-5,000 μM, and thus on the basis of these results, it was confirmed that osmundacetone had great safety when used as a medicine.

### Application Example 1

Osteoporosis

Bone is maintained through a balanced bone remodeling cycle between osteoclasts that metabolically resorb bones and osteoblasts that form bones. However, when osteoclasts are extremely activated due to the destruction of the balance between osteoclasts and osteoblasts, the balance between bone resorption and formation is destroyed, and thus the amount of bone uptake is greater than the amount of bone formation, causing osteoporosis (Kim J H and Kim N, 2016; Shiozawa Y et al., 2011).

Therefore, osmundacetone of the present invention simultaneously shows an effect of inhibiting the proliferation and differentiation of osteoclasts and an effect of activating osteoblasts, and thus can exhibit a preventive or therapeutic effect on osteoporosis.

### Application Example 2

Rheumatoid Arthritis

Rheumatoid arthritis is an autoimmune disease, and autoimmune antibodies promote osteoclast differentiation. The resultant excessive bone resorption worsens rheumatoid arthritis (Takayanagi H, 2007). The mechanism thereof is as follows. NFAT transcription factors (NFATc1/c2/c3/c4), which are key transcription factors related to osteoclast differentiation, are basically activated by calcium/calmodulin signaling (Takayanagi H et al., 2002). For complete activation, tyrosine-based activation motif (ITAM)-bearing molecules, such as the immunoregulatory protein DNAX-activating protein 12 (DAP12) and the immune antibody Fc receptor common γ chain (FcRγ), stimulates calcium signaling in immune cells (Pitcher L A and van Oers N S, 2003). Also, DAP12 and FcRγ activate NFATc1 through calcium signaling in osteoclasts. Therefore, immunoglobulin-like receptors involved in DAP12 and FcRγ play a key role in the differentiation of osteoclasts (Koga T et al., 2004; Mocsai A et al., 2004). That is, FcRγ interacts with osteoclast-associated receptor (OSCAR) and paired immunoglobulin-like receptor (PIR-A) in osteoclasts. The phosphorylation of ITAM activates phospholipase Cy (PLCγ), which is advantageous in the intracellular calcium, which activates calcineurin, which is calmodulin-dependent phosphatase. Calcineurin directly dephosphorylates serine of NFATc1, resulting in translocation into the nucleus, and activates NFATc1. Resultingly, the immune antibodies promote osteoclast differentiation, and excessive bone resorption by osteoclasts worsens rheumatoid arthritis. Ultimately, in rheumatoid arthritis patients, the inhibition of osteoclast differentiation cannot correct the abnormality of the autoimmune mechanism per se, but can treat skeletal symptoms, such as arthritis and pain resulting therefrom.

Therefore, osmundacetone of the present invention simultaneously shows an effect of inhibiting the proliferation and differentiation of osteoclasts, and thus can exhibit a preventive or therapeutic effect on rheumatoid arthritis.

### Application Example 3

Paget's Disease (Osteitis deformans)

Paget's disease (Osteitis deformans) is also caused by abnormal bone resorption of osteoclasts (Singer F R, 2016). Therefore, abnormal osteogenesis of osteoblasts progresses, and this process is repeated, resulting in bone malformation, causing pains, headache, hearing loss, or the like, resulting therefrom. Paget's disease is frequently caused in arms, legs, pelvis, spine, and skull. The newly formed bone is weak, and thus the frequency of fracture is high. Hypercalcemia, heart attack, and hemiparesis may be caused (Ralstone S H, 2016). The cause of the disease is unknown, but genetic susceptibility and childhood virus infection are suspected to be the cause. The medication treatment is helpful in inhibiting the progression of the disease. Fosamax, an osteoclast differentiation inhibitor, and calcitonin, which regulates bone metabolism, are currently the most commonly used medicines. However, Fosamax is restricted in long-term use in some patients due to side effects thereof. Acetaminophen (Tylenol) or nonsteroidal anti-inflammatory drugs (NSAIDs) are used for severe pains.

Therefore, osmundacetone of the present invention shows an effect of inhibiting the proliferation and differentiation of osteoclasts, and thus can exhibit a preventive or therapeutic effect on Paget's disease.

### Application Example 4

Bone Metastatic Cancer

Osteoclasts also promote bone metastasis of solid tumor. Bone is the most frequent site of cancer metastasis. The metastasis of cancer to bone causes severe pains and bone fractures, thereby significantly reducing the possibility of complete cure (Weilbaecher K N et al., 2011). Cancer cells spread throughout the body are found in proliferation sites of blood stem cells in the bone marrow (Shiozawa Y et al., 2013). Cancer cells significantly promote the differentiation of osteoclasts from bone marrow cells, thereby promoting bone metastasis, cancer growth, and bone destruction. Therefore, osteoclasts play a key role in the bone metastasis of cancer, and the inhibition of osteoclast differentiation reduces bone metastasis. Many solid cancer metastases correspond to bone metastasis, and blood stem cells are driven and grown based on blood stem cell proliferation sites, and then again comes into the blood, and metastasized to a different site. In prostate cancer, bone metastasis occurs most frequently, and such bone metastasis worsens cancer to make the cure of cancer difficult, resulting in a major cause of death. The direct main target of human prostate cancer cells is also a proliferation site of blood stem cells, and used as a key base of metastatic cancer (Shiozawa Y et al., 2011). In addition, osteoclasts promote angiogenesis in prostate cancer tissues, thereby promoting cancer growth (Bruni-Cardoso A et al., 2010). Breast cancer cells also promote osteoclast differentiation, and thus osteoclasts promote cancer recurrence through bone metastasis in breast cancer patients undergoing mastectomy (Danilin S et al., 2012; Lu X et al., 2011).

Bone-targeting therapeutic agents to prevent bone metastatic cancer are currently being used in clinical practice. Osteoclasts are one of the key mechanisms of bone metastasis of cancer, and thus become a major target of development of new anti-cancer drugs. Zoledronic acid is currently the only bisphosphonate-based drug, approved by the US FDA, for the purpose of inhibiting osteoclast differentiation (El-Amm J et al., 2013). Zoledronic acid preserves bones and increases survival rates. Zoledronic acid significantly reduced bone metastasis in high risk nonmetastatic prostate cancer (Wirth M et al., 2014). The co-administration of zoledronic acid with parathyroid hormone that activates osteoblasts further reduced bone metastasis (Schneider A et al., 2005). It was again verified that denosumab, a monoclonal antibody to RANKL, a signaling substance for osteoclast differentiation, also inhibited bone metastasis of prostate cancer, and thus the osteoclast inhibition is important for inhibiting bone metastasis of cancer (Smith M R et al., 2012). The administration of zoledronic acid inhibited osteoclast differentiation, thereby significantly inhibiting bone metastasis, even in patients with multiple myeloma (Zhuang J et al., 2012). That is, if an osteoclast inhibitor having few side effects and low cost is developed, such osteoclast inhibitor can be administered for a long time to inhibit metastasis in cancer patients.

Therefore, osmundacetone of the present invention simultaneously shows an effect of inhibiting the proliferation and differentiation of osteoclasts and an effect of activating osteoblasts, and thus can exhibit a preventive or therapeutic effect on bone metastatic cancer.

### REFERENCES

Anderson D M, Maraskovsky E, Billingsley W L, Dougall W C, Tometsko M E, Roux E R, et al. A homologue of the TNF receptor and its ligand enhance T-cell growth and dendritic-cell function. Nature 1997; 390:175-9.
Bonewald, L. F., et al., Leukotriene B4 stimulates osteoclastic bone resorption both in vitro and in vivoJ. Bone Miner. Res., 11, 1619-1627, 1996.
Bonewald, L. F. et al., Effects of synthetic peptido-leukotrienes on bone resorption in vitroJ. Bone Miner. Res., 11, 521-529, 1996.
Bruni-Cardoso A, Johnson L C, Vessella R L, Peterson T E, Lynch C C. Osteoclast-derived matrix metalloproteinase-9 directly affects angiogenesis in the prostate tumor-bone microenvironment. Mol Cancer Res. 2010 April; 8(4):459470. PMCID: PMC2946627
Danilin S, Merkel A R, Johnson J R, Johnson R W, Edwards J R, Sterling J A. Myeloid-derived suppressor cells expand during breast cancer progression and promote tumor-induced bone destruction. oncoimmunology. 2012 Dec. 1; 1(9):14841494.
Darnay B G, Haridas V, Ni J, Moore P A, Aggarwal B B. Characterization of the intracellular domain of receptor activator of NF-kappaB (RANK). Interaction with tumor necrosis factor receptor-associated factors and activation of NF-kappab and c-Jun N-terminal kinase. J Biol Chem 1998; 273:20551-5.
Dougall W C, Glaccum M, Charrier K, Rohrbach K, Brasel K, De Smedt T, et al. RANK is essential for osteoclast and lymph node development. Genes Dev 1999; 13:2412-24.
El-Amm J, Freeman A, Patel N, Aragon-Ching J B. Bone-targeted therapies in metastatic castration-resistant prostate cancer: evolving paradigms. Prostate Cancer. Hindawi Publishing Corporation; 2013; 2013:210686. PMCID: PMC3771418
Ford-Hutchinson, A. W., et al., Leukotriene B, a potent chemokinetic and aggregating substance released from polymorphonuclear leukocytes. Nature (London), 286, 264-265, 1980.
Kim J H and Kim N. Signaling Pathways in Osteoclast Differentiation. Chonnam Med J 2016; 52:12-17.
Kobayashi N, Kadono Y, Naito A, Matsumoto K, Yamamoto T, Tanaka S, et al. Segregation of TRAF6-mediated signaling pathways clarifies its role in osteoclastogenesis. EMBO J 2001; 20:1271-80.
Koga T, Inui M, Inoue K, Kim S, Suematsu A, Kobayashi E, et al. Costimulatory signals mediated by the ITAM motif cooperate with RANKL for bone homeostasis. Nature 2004; 428:758-63.
Kong Y Y, Yoshida H, Sarosi I, Tan H L, Timms E, Capparelli C, et al. OPGL is a key regulator of osteoclastogenesis, lymphocyte development and lymph-node organogenesis. Nature 1999; 397: 315-23.
Lacey D L, Timms E, Tan H L, Kelley M J, Dunstan C R, Burgess T, et al. Osteoprotegerin ligand is a cytokine that regulates osteoclast differentiation and activation. Cell 1998; 93:165-76.
Lee Sung-eun, Synthesis and Biological Activity of Natural Products and Designed New Hybrid Compounds for the Treatment of LTB4 Related Disease, Ph. D thesis, Graduate School of Pusan Univ., August 1999
Lomaga M A, Yeh W C, Sarosi I, Duncan G S, Furlonger C, Ho A, et al. TRAF6 deficiency results in osteopetrosis and defective interleukin-1, CD40, and LPS signaling. Genes Dev 1999; 13: 1015-24.
Lum L, Wong B R, Josien R, Becherer J D, Erdjument-Bromage H, SchloJ, et al. Evidence for a role of a tumor necrosis factor-alpha (TNF-alpha)-converting enzyme-like protease in shedding of TRANCE, a TNF family member involved in osteoclastogenesis and dendritic cell survival. J Biol Chem 1999; 274: 13613-8.
Lu X, Mu E, Wei Y, Riethdorf S, Yang Q, Yuan M, Yan J, Hua Y, Tiede B J, Lu X, Haffty B G, Pantel K, MassaguE J, Kang Y. VCAM-1 promotes osteolytic expansion of indolent bone micrometastasis of breast cancer by engaging a431-positive osteoclast progenitors. Cancer Cell. 2011 Dec. 13; 20(6):701714. PMCID: PMC3241854
Mancini A, Niedenthal R, Joos H, Koch A, Trouliaris S, Niemann H, et al. Identification of a second Grb2 binding site in the v-Fms tyrosine kinase. Oncogene 1997; 15:1565-72.
MoA, Humphrey M B, Van Ziffle J A, Hu Y, Burghardt A, Spusta S C, et al. The immunomodulatory adapter proteins DAP12 and Fc receptor gamma-chain (FcRgamma) regulate development of functional osteoclasts through the Syk tyrosine kinase. Proc Natl Acad Sci USA 2004; 101:6158-63.
Molina M'ICREA V R-G, PARDO-DE-SANTAYANA M. Local Knowledge and Management of the Royal Fern (Osmunda regalis L.) in Northern Spain: Implications for Biodiversity Conservation. American Fern Journal 2009; 99(1):4555
Mundy, G. R., et al., 5-Lipoxygenase metabolites of arachidonic acid stimulate isolated osteoclasts to resorb calcified matrices. J. Bio. Chem., 268, 10087-10094, 1993.
Naito A, Azuma S, Tanaka S, Miyazaki T, Takaki S, Takatsu K, et al. Severe osteopetrosis, defective interleukin-1 signalling and lymph node organogenesis in TRAF6-deficient mice. Genes Cells 1999; 4:353-62.
Pitcher L A, van Oers N S. T-cell receptor signal transmission: who gives an ITAM—Trends Immunol 2003; 24:554-60.
Ralston S H. Paget disease of bone. In: Goldman L, Schafer A I, eds. Goldman's Cecil Medicine. 25th ed. Philadelphia, Pa.: Elsevier Saunders; 2016: chap 247.
Schneider A, Kalikin L M, Mattos A C, Keller E T, Allen M J, Pienta K J, McCauley L K. Bone turnover mediates preferential localization of prostate cancer in the skeleton. Endocrinology. 2005 April; 146(4):17271736. PMID:
Sherr C J. Colony-stimulating factor-1 receptor. Blood 1990; 75: 1-12.
Shiozawa Y, Pedersen E A, Havens A M, Jung Y, Mishra A, Joseph J, Kim J K, Patel L R, Ying C, Ziegler A M, Pienta M J, Song J, Wang J, Loberg R D, Krebsbach P H, Pienta K J, Taichman R S. Human prostate cancer metastases target the hematopoietic stem cell niche to establish footholds in mouse bone marrow. J Clin Invest. 2011 April; 121(4):12981312. PMCID: PMC3069764.
Shiozawa Y, Taichman R S, Keller E T. Detection and isolation of human disseminated tumor cells in the murine bone marrow stem cell niche. Methods Mol Biol. Humana Press; 2013; 1035:207215. PMID: 23959994.
Singer F R. Paget's disease of bone. In: Jameson J L, De Groot L J, de Krester D M, et al, eds. Endocrinology: Adult and Pediatric. 7th ed. Philadelphia, Pa.: Elsevier Saunders; 2016: chap 72.
Smith M R, Saad F, Coleman R, Shore N, Fizazi K, Tombal B, Miller K, Sieber P, Karsh L, DamiaR, Tammela T L, Egerdie B, Van Poppel H, Chin J, Morote J, GoF, Borkowski T, Ye Z, Kupic A, Dansey R, Goessl C. Denosumab and bone-metastasis-free survival in men with castration-resistant prostate cancer: results of a phase 3, randomised, placebo-controlled trial. Lancet. 2012 Jan. 7; 379(9810):3946. PMCID: PMC3671878
Suda T, Takahashi N, Udagawa N, Jimi E, Gillespie M T, Martin T J. Modulation of osteoclast differentiation and function by the new members of the tumor necrosis factor receptor and ligand families. Endocr Rev 1999; 20:345-57.
Takayanagi H, Kim S, Koga T, Nishina H, Isshiki M, Yoshida H, et al. Induction and activation of the transcription factor NFATc1 (NFAT2) integrate RANKL signaling in terminal differentiation of osteoclasts. Dev Cell 2002; 3:889-901.
Takayanagi H. Osteoimmunology: shared mechanisms and crosstalk between the immune and bone systems. Nat Rev Immunol 2007; 7:292-304
Takayanagi H, Kim S, Koga T, Nishina H, Isshiki M, Yoshida H, et al. Induction and activation of the transcription factor NFATc1 (NFAT2) integrate RANKL signaling in terminal differentiation of osteoclasts. Dev Cell 2002; 3:889-901.
Teitelbaum S L, Ross F P. Genetic regulation of osteoclast development and function. Nat Rev Genet 2003; 4:638-49.
Walsh M C, Choi Y. Biology of the TRANCE axis. Cytokine Growth Factor Rev 2003; 14:251-63.
Weilbaecher K N, Guise T A, McCauley L K. Cancer to bone: a fatal attraction. Nat Rev Cancer. 2011 June; 11(6):411425. PMID: 21593787
Wirth M, Tammela T, Cicalese V, Veiga F G, Delaere K, Miller K, Tubaro A, Schulze M, Debruyne F, Huland H, Patel A, Lecouvet F, Caris C, Witjes W. Prevention of Bone Metastases in Patients with High-risk Nonmetastatic Prostate Cancer Treated with Zoledronic Acid: Efficacy and Safety Results of the Zometa European Study (ZEUS). European Urology. European Association of Urology; 2014 Mar. 11; 110.
Wong B R, Besser D, Kim N, Arron J R, Vologodskaia M, Hanafusa H, et al. TRANCE, a TNF family member, activates Akt/PKB through a signaling complex involving TRAF6 and c-Src. Mol Cell 1999; 4:1041-9.
Wong B R, Josien R, Lee S Y, Vologodskaia M, Steinman R M, Choi Y. The TRAF family of signal transducers mediates NF-kappaB activation by the TRANCE receptor. J Biol Chem 1998; 273: 28355-9.
Zhuang J, Zhang J, Lwin S T, Edwards J R, Edwards C M. Osteoclasts in multiple myeloma are derived from Gr-1+CD11b+myeloid-derived suppressor cells. Shi S, editor. PLoS One. 2012.

### INDUSTRIAL APPLICABILITY

The compositions according to the present invention show strong inhibitory effects on osteoclast proliferation and differentiation and, simultaneously, activate osteoblasts, and thus can be favorably used to develop a safe and effective osteoporosis medicine or a safe and effective food for alleviating osteoporosis.

1-9. (canceled)

10. A method for treating a bone disease in a subject, the method comprising administering an effective amount of a composition comprising osmundacetone or a pharmaceutically acceptable salt thereof as an active ingredient to a subject in need thereof, wherein the bone disease is at least one selected from the group consisting of osteoporosis, rheumatoid arthritis, arthralgia, Paget's disease, bone metastatic cancer, and bone fractures.

11-12. (canceled)

13. A method for treating a bone disease in a subject, the method comprising administering an effective amount of a composition comprising an Osmunda japonica extract as an active ingredient to a subject in need thereof, wherein the bone disease is at least one selected from the group consisting of osteoporosis, rheumatoid arthritis, arthralgia, Paget's disease, bone metastatic cancer, and bone fractures.

14. The method of claim 13, wherein the extract is extracted with at least one solvent selected from the group consisting of water, ethanol, grain ethanol, methanol, propanol, isopropanol, butanol, acetone, ether, chloroform, ethyl acetate, methylene chloride, hexane, cyclohexane, petroleum ether, diethyl ether, and benzene.

15. The method of claim 10, wherein the composition is a pharmaceutical composition or a food composition.

16. The method of claim 10, wherein the osmundacetone is isolated from a plant in the family Osmundaceae.

17. The method of claim 16, wherein the plant in the family Osmundaceae is Osmunda japonica.

18. The method of claim 13, wherein the composition is a pharmaceutical composition or a food composition.
